SEQUENCE LISTING (25 sequences)

SEQ ID NO: 1 - protein, 9 aa, synthetic construct
PPPPPPPPP

SEQ ID NO: 2 - protein, 9 aa, synthetic construct
  ACT_SITE 1: note = the derivative 1 of proline
XPPPPPPPP

SEQ ID NO: 3 - protein, 9 aa, synthetic construct
  ACT_SITE 1: note = the derivative 1 of proline
  ACT_SITE 9: note = the derivative 2 of proline
XPPPPPPPX

SEQ ID NO: 4 - protein, 9 aa, synthetic construct
  ACT_SITE 1: note = the derivative 3 of proline
  ACT_SITE 8: note = the derivative 4 of proline
  ACT_SITE 9: note = the derivative 2 of proline
XPPPPPPXX

SEQ ID NO: 5 - protein, 9 aa, synthetic construct
  ACT_SITE 1: note = the derivative 3 of proline
  ACT_SITE 2: note = the derivative 5 of proline
  ACT_SITE 8: note = the derivative 4 of proline
  ACT_SITE 9: note = the derivative 2 of proline
XXPPPPPXX

SEQ ID NO: 6 - protein, 9 aa, synthetic construct
  ACT_SITE 1: note = the derivative 3 of proline
  ACT_SITE 8: note = the derivative 6 of proline
  ACT_SITE 9: note = the derivative 2 of proline
XPPPPPPXX

SEQ ID NO: 7 - protein, 9 aa, synthetic construct
  ACT_SITE 1: note = the derivative 3 of proline
  ACT_SITE 2: note = the derivative 5 of proline
  ACT_SITE 8: note = the derivative 6 of proline
  ACT_SITE 9: note = the derivative 2 of proline
XXPPPPPXX

SEQ ID NO: 8 - protein, 9 aa, synthetic construct
  ACT_SITE 1: note = the derivative 3 of proline
  ACT_SITE 2: note = the derivative 6 of proline
  ACT_SITE 8: note = the derivative 5 of proline
  ACT_SITE 9: note = the derivative 2 of proline
XXPPPPPXX

SEQ ID NO: 9 - protein, 9 aa, synthetic construct
  ACT_SITE 1: note = the derivative 3 of proline
  ACT_SITE 2: note = the derivative 4 of proline
  ACT_SITE 9: note = the derivative 2 of proline
XXPPPPPPX

SEQ ID NO: 10 - protein, 27 aa, synthetic construct
  ACT_SITE 8: note = the derivative 6 of proline
  ACT_SITE 20: note = the derivative 5 of proline
  ACT_SITE 17: note = the derivative 4 of proline
  ACT_SITE 26: note = the derivative 4 of proline
PPPPPPPXPPPPPPPPXPPXPPPPPXP

SEQ ID NO: 11 - protein, 27 aa, synthetic construct
  ACT_SITE 2: note = the derivative 5 of proline
  ACT_SITE 8: note = the derivative 6 of proline
  ACT_SITE 17: note = the derivative 4 of proline
  ACT_SITE 26: note = the derivative 4 of proline
PXPPPPPXPPPPPPPPXPPPPPPPPXP

SEQ ID NO: 12 - protein, 27 aa, synthetic construct
  ACT_SITE 2: note = the derivative 6 of proline,
  ACT_SITE 8: note = the derivative 5 of proline,
  ACT_SITE 11: note = the derivative 4 of proline,
  ACT_SITE 20: note = the derivative 4 of proline,
PXPPPPPXPPXPPPPPPPPXPPPPPPP

SEQ ID NO: 13 - protein, 27 aa, synthetic construct
  ACT_SITE 20: note = the derivative 5 of proline
PPPPPPPPPPPPPPPPPPPXPPPPPPP

SEQ ID NO: 14 - protein, 27 aa, synthetic construct
  ACT_SITE 8: note = the derivative 5 of proline
PPPPPPPXPPPPPPPPPPPPPPPPPPP

SEQ ID NO: 15 - protein, 27 aa, synthetic construct
  ACT_SITE 2: note = the derivative 5 of proline
PXPPPPPPPPPPPPPPPPPPPPPPPPP

SEQ ID NO: 16 - protein, 9 aa, synthetic construct
  ACT_SITE 1: note = the derivative 1 of proline
  ACT_SITE 6: note = the derivative 5 of proline
XPPPPXPPP

SEQ ID NO: 17 - protein, 9 aa, synthetic construct
  ACT_SITE 1: note = the derivative 1 of proline
  ACT_SITE 2: note = the derivative 4 of proline
XXPPPPPPP

SEQ ID NO: 18 - protein, 18 aa, synthetic construct
  ACT_SITE 2: note = the derivative 5 of proline
  ACT_SITE 8: note = the derivative 5 of proline
  ACT_SITE 16: note = the derivative 4 of proline
  ACT_SITE 14: note = the derivative 5 of proline
PXPPPPPXPPPPPXPXPP

SEQ ID NO: 19 - protein, 18 aa, synthetic construct
  ACT_SITE 3: note = the derivative 5 of proline
  ACT_SITE 9: note = the derivative 5 of proline
  ACT_SITE 16: note = the derivative 4 of proline
  ACT_SITE 15: note = the derivative 5 of proline
PPXPPPPPXPPPPPXXPP

SEQ ID NO: 20 - protein, 18 aa, synthetic construct
  ACT_SITE 2: note = the derivative 7 of proline
  ACT_SITE 8: note = the derivative 7 of proline
  ACT_SITE 16: note = the derivative 10 of proline
  ACT_SITE 14: note = the derivative 7 of proline
PXPPPPPXPPPPPXPXPP

SEQ ID NO: 21 - protein, 18 aa, synthetic construct
  ACT_SITE 2: note = the derivative 8 of proline
  ACT_SITE 8: note = the derivative 8 of proline
  ACT_SITE 16: note = the derivative 10 of proline
  ACT_SITE 14: note = the derivative 8 of proline
PXPPPPPXPPPPPXPXPP

SEQ ID NO: 22 - protein, 18 aa, synthetic construct
  ACT_SITE 2: note = the derivative 9 of proline
  ACT_SITE 8: note = the derivative 9 of proline
  ACT_SITE 16: note = the derivative 10 of proline
  ACT_SITE 14: note = the derivative 9 of proline
PXPPPPPXPPPPPXPXPP

SEQ ID NO: 23 - protein, 18 aa, synthetic construct
  ACT_SITE 3: note = the derivative 7 of proline
  ACT_SITE 9: note = the derivative 7 of proline
  ACT_SITE 16: note = the derivative 10 of proline
  ACT_SITE 15: note = the derivative 7 of proline
PPXPPPPPXPPPPPXXPP

SEQ ID NO: 24 - protein, 18 aa, synthetic construct
  ACT_SITE 3: note = the derivative 8 of proline
  ACT_SITE 9: note = the derivative 8 of proline
  ACT_SITE 16: note = the derivative 10 of proline
  ACT_SITE 15: note = the derivative 8 of proline
PPXPPPPPXPPPPPXXPP

SEQ ID NO: 25 - protein, 18 aa, synthetic construct
  ACT_SITE 3: note = the derivative 9 of proline
  ACT_SITE 9: note = the derivative 9 of proline
  ACT_SITE 16: note = the derivative 10 of proline
  ACT_SITE 15: note = the derivative 9 of proline
PPXPPPPPXPPPPPXXPP

(19) United States
(12) Patent Application Publication
(10) Pub. No.: US 2026/0085133 A1
(43) Pub. Date: Mar. 26, 2026

(54) POLYPROLINE NANOCAGE COMPOUND AND MANUFACTURING METHOD THEREOF

(71) Applicant: NATIONAL TSING HUA UNIVERSITY, Hsinchu (TW)

(72) Inventors: Sheng-Kai WANG, HSINCHU (TW); Chia-Lung TSAI, HSINCHU (TW); Yi-Cheng HSU, HSINCHU (TW); Hsiao-Ling CHIU, HSINCHU (TW); Tzu-Yuan CHEN, HSINCHU (TW)

(21) Appl. No.: 19/338,380

(22) Filed: Sep. 24, 2025

(30) Foreign Application Priority Data
Sep. 26, 2024 (TW) 113136706

(51) Int. Cl. C07K 19/00; C07K 14/00

(52) U.S. Cl. CPC C07K 19/00; C07K 14/001

(57) ABSTRACT

A polyproline nanocage compound includes two cyclic peptide scaffolds and at least three connecting molecules. The two cyclic peptide scaffolds include at least three polyproline helix rods and at least three turn-angle molecules. Each of the at least three polyproline helix rods is composed of a plurality of repeat units. Two of the at least three polyproline helix rods are connected by one of the at least three turn-angle molecules so as to form a closed ring. The at least three connecting molecules are respectively connected from the at least three polyproline helix rods of one of the two cyclic peptide scaffolds to the at least three polyproline helix rods of the other of the two cyclic peptide scaffolds.

### RELATED APPLICATIONS

This application claims priority to Taiwan Application Serial Number 113136706, filed Sep. 26, 2024, which is herein incorporated by reference.

### SEQUENCE LISTING XML

A sequence listing XML submitted as an xml file via EFS-WEB is incorporated herein by reference. The sequence listing XML file submitted via EFS-WEB with the name “CP-6529-US_SEQ_LIST” was created on Sep. 9, 2025, which is 51,811 bytes in size.

### BACKGROUND

### Technical Field

The present disclosure relates to a nanocage compound and a manufacturing method thereof. More particularly, the present disclosure relates to a polyproline nanocage compound and a manufacturing method thereof.

### Description of Related Art

Cage compounds are molecules with three-dimensional and rigid geometric structures, and scaffolds of the cage compounds are mainly composed of carbon-carbon bones and other common functional groups. The cage compounds generally have specific internal structures, sizes capable to host other molecules, structures that do not easily collapse or distort, and shapes approximate to a geometric shape, such as tetrahedron, cube or octahedron, etc.

In recent years, many applications of the cage compounds are developed, include recognizing and separating ions or molecules, whose molecular size can be as large as C₆₀ or oligosaccharides. Furthermore, the cage compounds can be used to protect compounds unstable at room temperature, and can even be drug-carriers. Currently, some cage compounds are assembled through metal complexes or can use aromatic rings as major frameworks. When the aforementioned cage compounds are applied in vivo, the metal complexes or the aromatic rings may become toxic through the metabolism. Other cage compounds are assembled by biomolecules (such as DNA, etc.) recognizing each other, and the volumes of the cage compounds with biomolecules are often very large. Moreover, the units of the aforementioned cage compounds are not connected to each other with covalent bonds, which may fall apart and lose effects in complicated environments.

For all these reasons, it is necessary to design a biocompatible cage compound that can control the connection of multiple functional groups to specific positions of the cage compounds so as to be suitable for various applications.

### SUMMARY

According to one aspect of the present disclosure, a polyproline nanocage compound includes two cyclic peptide scaffolds and at least three connecting molecules. Each of the two cyclic peptide scaffolds includes at least three polyproline helix rods and at least three turn-angle molecules. Each of the at least three polyproline helix rods is composed of a plurality of repeat units, and each of the plurality of repeat units is represented by Formula (i):

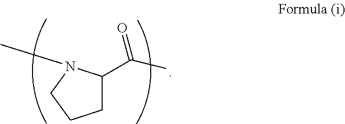

Two of the at least three polyproline helix rods are connected by one of the at least three turn-angle molecules so as to form a closed ring. The at least three connecting molecules are respectively connected from the at least three polyproline helix rods of one of the two cyclic peptide scaffolds to the at least three polyproline helix rods of the other of the two cyclic peptide scaffolds.

According to another aspect of the present disclosure, a polyproline nanocage compound includes a cyclic peptide scaffold and at least three connecting molecules. The cyclic peptide scaffold includes at least three polyproline helix rods and at least three turn-angle molecules. Each of the at least three polyproline helix rods is composed of a plurality of repeat units, and each of the plurality of repeat units is represented by Formula (i):

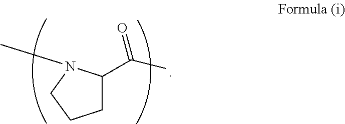

Two of the at least three polyproline helix rods are connected by one of the at least three turn-angle molecules so as to form a closed ring. One terminus of each of the at least three connecting molecules is connected to one of the at least three polyproline helix rods of the cyclic peptide scaffold, and the other terminus of each of the at least three connecting molecules is connected to the other terminus of another of the at least three connecting molecules.

According to still another aspect of the present disclosure, a manufacturing method of a polyproline nanocage compound includes providing two cyclic peptide scaffolds, performing a de-protecting step, performing a first cycloaddition, performing a second cycloaddition, and performing a connecting step. Each of the two cyclic peptide scaffolds includes at least three polyproline helix rods, at least three turn-angle molecules and at least three modifying functional groups. Each of the at least three polyproline helix rods is composed of a plurality of repeat units, and each of the plurality of repeat units is represented by Formula (i):

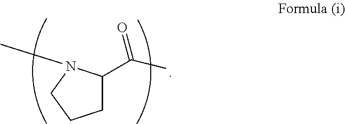

Two of the at least three polyproline helix rods are connected by one of the at least three turn-angle molecules so as to form a closed ring. The at least three modifying functional groups are respectively connected to the at least three polyproline helix rods, and each of the at least three modifying functional groups is formed by protecting an amino group with a protecting group. In the de-protecting step, the protecting group of one of the at least three modifying functional groups of each of the two cyclic peptide scaffolds is removed to form a first primary amine, and an azide compound is added so that the first primary amine is converted into a first azido group. In the first cycloaddition, a first connecting intermediate with an alkynyl group at one terminus and with a carboxyl group at the other terminus is catalyzed via a Cu ion, so that the alkynyl group of the first connecting intermediate is connected to the first azido group of one of the two cyclic peptide scaffolds. In the second cycloaddition, the carboxyl group of the first connecting intermediate is modified to form an alkynyl terminus, so that the alkynyl terminus is connected to the first azido group of the other of the two cyclic peptide scaffolds. In the connecting step, the protecting groups of another at least two of the at least three modifying functional groups of each of the two cyclic peptide scaffolds are removed to respectively form at least two second primary amines, the at least two second primary amines are converted into at least two second azido groups, at least two second connecting intermediates with the alkynyl groups at two termini are catalyzed via the Cu ion, so that the at least two second azido groups of each of the two cyclic peptide scaffolds are connected to the alkynyl groups of the at least two second connecting intermediates so as to form a polyproline nanocage compound. At least three connecting molecules are respectively formed from the first connecting intermediate after performing the second cycloaddition and from the at least two second connecting intermediates after performing the connecting step.

According to yet another aspect of the present disclosure, a manufacturing method of a polyproline nanocage compound includes providing a cyclic peptide scaffold, providing at least three first connecting intermediates, performing a first de-protecting step, performing a first cycloaddition, performing a second de-protecting step, performing a second cycloaddition, and performing a connecting step. The cyclic peptide scaffold includes at least three polyproline helix rods, at least three turn-angle molecules and at least three modifying functional groups. Each of the at least three polyproline helix rods is composed of a plurality of repeat units, and each of the plurality of repeat units is represented by Formula (i):

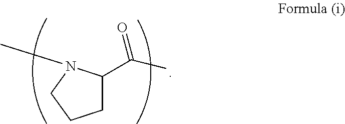

Two of the at least three polyproline helix rods are connected by one of the at least three turn-angle molecules so as to form a closed ring. The at least three modifying functional groups are respectively connected to the at least three polyproline helix rods, and each of the at least three modifying functional groups is formed by protecting an amino group with a protecting group. Each of the at least three first connecting intermediates is with an alkynyl group at one terminus and with a carboxyl group at the other terminus. In the first de-protecting step, the protecting group of one of the at least three modifying functional groups is removed to form a first primary amine, and an azide compound is added so that the first primary amine is converted into a first azido group. In the first cycloaddition, one of the at least three first connecting intermediates is catalyzed via a Cu ion, so that the alkynyl group of the one of the at least three first connecting intermediates is connected to the first azido group of the cyclic peptide scaffold. In the second de-protecting step, the protecting groups of another at least two of the at least three modifying functional groups of the cyclic peptide scaffold are removed to form at least two second primary amines, so that the at least two second primary amines are converted into at least two second azido groups. In the second cycloaddition, another at least two of the at least three first connecting intermediates are catalyzed via the Cu ion, so that the alkynyl groups of the another at least two of the at least three first connecting intermediates are connected to the at least two second azido groups of the cyclic peptide scaffold. In the connecting step, the carboxyl groups of the at least three first connecting intermediates are modified to azido groups, and are connected to each other so as to form a polyproline nanocage compound. At least three first connecting molecules are formed from the at least three first connecting intermediates after performing the connecting step.

### BRIEF DESCRIPTION OF THE DRAWINGS

The present disclosure can be more fully understood by reading the following detailed description of the embodiment, with reference made to the accompanying drawings as follows:

FIG. 1 is a schematic view of a polyproline nanocage compound according to one embodiment of the present disclosure.

FIG. 2 is a schematic view of a polyproline nanocage compound according to another embodiment of the present disclosure.

FIG. 3 is a flow chart of a manufacturing method of a polyproline nanocage compound according to still another embodiment of the present disclosure.

FIG. 4 is a schematic process diagram of the manufacturing method of the polyproline nanocage compound according to FIG. 3.

FIG. 5 is a flow chart of a manufacturing method of a polyproline nanocage compound according to yet another embodiment of the present disclosure.

FIG. 6 is a schematic process diagram of the manufacturing method of the polyproline nanocage compound according to FIG. 5.

FIG. 7A is a manufacturing process diagram of a polyproline nanocage compound according to Example 1 and Example 2.

FIG. 7B is a manufacturing process diagram of a polyproline nanocage compound according to Example 3.

FIG. 7C is a manufacturing process diagram of a polyproline nanocage compound according to Example 4.

### DETAILED DESCRIPTION

The present disclosure will be further exemplified by the following specific embodiments. However, the embodiments can be applied to various inventive concepts and can be embodied in various specific ways. The specific embodiments are only for the purposes of description, and are not limited to these practical details thereof.

The term “X” represents different derivatives of proline, and the derivatives of proline are summarized in Table A.

TABLE A

| Derivative | Structural formula |
|---|---|
| 1 | 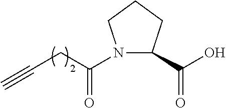 |
| 2 | 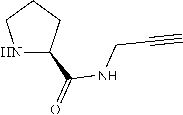 |
| 3 | 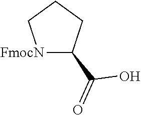 |
| 4 | 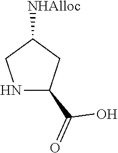 |
| 5 | 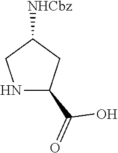 [SMILES: O=C(N[C@H]1CN[C@H](C(=O)O)C1)OCc1ccccc1] |
| 6 | 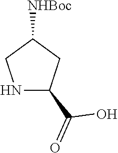 [SMILES: CC(C)(C)OC(=O)N[C@H]1CN[C@H](C(=O)O)C1] |
| 7 | 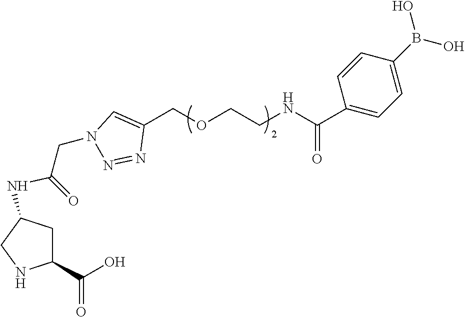 |
| 8 | 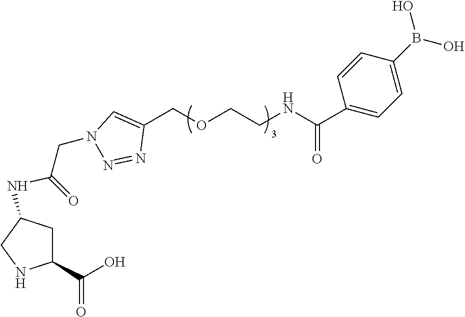 |
| 9 | 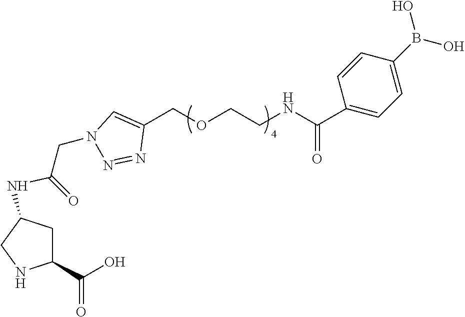 |
| 10 | 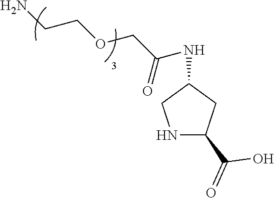 |

### <Polyproline Nanocage Compound>

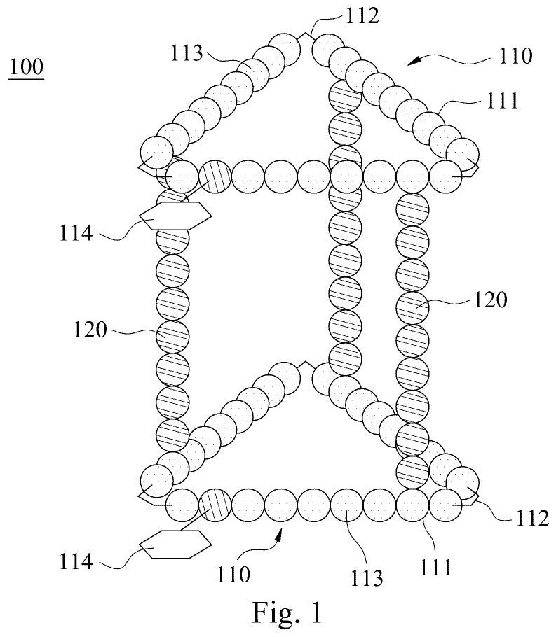

Reference is made to FIG. 1, which is a schematic view of a polyproline nanocage compound 100 according to one embodiment of the present disclosure. In FIG. 1, the polyproline nanocage compound 100 includes two cyclic peptide scaffolds 110 and at least three connecting molecules 120.

In detail, each of the two cyclic peptide scaffolds 110 includes at least three polyproline helix rods 111 and at least three turn-angle molecules 112, two of the at least three polyproline helix rods 111 are connected by one of the at least three turn-angle molecules 112 so as to form a closed ring, and each of the at least three polyproline helix rods 111 is composed of a plurality of repeat units 113, and each of the repeat units 113 is represented by Formula (i):

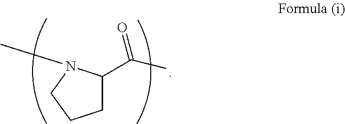

Moreover, the at least three connecting molecules 120 are respectively connected from the at least three polyproline helix rods 111 of one of the two cyclic peptide scaffolds 110 to the at least three polyproline helix rods 111 of the other of the two cyclic peptide scaffolds 110.

Each of the polyproline helix rods 111 can be a polyproline helix II rod, which is a structure formed from a polyproline peptide dissolved in a highly polar solvent, such as an aqueous solution, and the structure is stabilized by stereo electronic effect, so that the structure is not easily affected by hydrogen bonds in the environment. Furthermore, when each of the polyproline helix rods 111 is in polyproline helix II conformation, which is suitable as a research tool due to a good solubility in water, a high rigidity and a regularly helical structure. Further, the polyproline helix rod is suitable for biomedical applications, because there is no toxicity product after the decomposition of the polyproline helix rod.

Specifically, each of the polyproline helix rods 111 of the present disclosure is prepared via the solid phase peptide synthesis (SPPS), and a polymer resin is used as a solid support in the SPPS. The polymer resins will not be dissolved in organic solvents, and functional groups on the surfaces of the polymer resins can be connected to amino acid monomers, so that the amino acids with protecting groups at N-termini can be connected to the polymer resin. Moreover, the amino acid monomers can be connected in series through a series of de-protecting reactions and coupling reactions until that the target number of peptides residues is synthesized, and a specific condition can be used for the cleavage of the peptides from the polymer resins so as to produce desired peptide products.

Furthermore, a number of the repeat units 113 of each of the polyproline helix rods 111 can be but not limited to 4 to 12. Therefore, the cyclic peptide scaffolds 110 with different sizes can be synthesized to control the size of the polyproline nanocage compound 100 so as to host different guest molecules. Specifically, the number of the repeat units 113 of each of the polyproline helix rods 111 is 9 in FIG. 1, and each of the polyproline helix rods 111 can be represented by Formula (I). Each of the polyproline helix rods 111 is referenced as SEQ ID NO: 1.

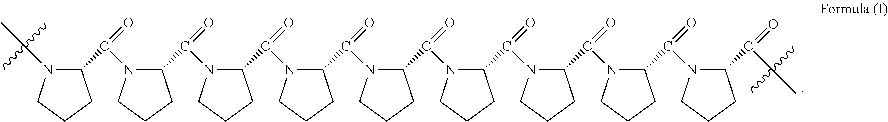

Each of the at least three turn-angle molecules 112 has a structure represented by Formula (ii):

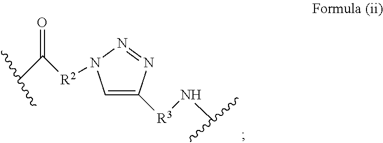

wherein, R² and R³ are independently a single bond or a divalent hydrocarbon group having 1 to 6 carbon atoms. Furthermore, the closed ring formed by the polyproline helix rods 111 and the turn-angle molecules 112 can be but not limited to a triangle, a quadrangle, a pentagon, a hexagon, a heptagon or an octagon, and the closed ring can provide an excellent spatial selectivity. For example, the number of the polyproline helix rods 111, the number of the turn-angle molecules 112 and the number of the connecting molecules 120 are all three in FIG. 1, and the closed ring formed by the cyclic peptide scaffolds 110 can be regarded as a triangle, so that the polyproline nanocage compound 100 formed by connecting the closed ring to the connecting molecules 120 is similar to a triangular prism.

The connecting molecules 120 are connected to the repeat units 113 of the polyproline helix rods 111, and the connecting molecules 120 can be connected to the repeat units 113 at different locations of the polyproline helix rods 111. In FIG. 1, each of the connecting molecules 120 is connected to the 8^{th} repeat unit 113 counted from the left to the right of each of the polyproline helix rods 111, but the present disclosure is not limited thereto. Furthermore, the connecting molecules 120 can be aliphatic hydrocarbon connecting molecules composed of small molecules or aromatic ring connecting molecules composed of small molecules. Alternately, a composition of the connecting molecules 120 can be the same as a composition of the polyproline helix rods 111, and the connecting molecules 120 has the repeat units 113 represented by Formula (i). Therefore, a structure of the polyproline nanocage compound 100 can be more stable, and a height of the polyproline nanocage compound 100 can be adjusted by changing the number of the repeat units of the connecting molecules 120, but the present disclosure is not limited thereto.

Moreover, in FIG. 1, at least one of the repeat units 113 of at least one of the at least three polyproline helix rods 111 of each of the two cyclic peptide scaffolds 110 can be connected to a functional group 114. When the composition of the connecting molecules 120 is the same as the composition of the polyproline helix rods 111, the repeat units of the connecting molecules 120 can be connected to functional groups. Therefore, different molecules or cells can be connected to certain positions, so that the polyproline nanocage compound 100 has a specific selectivity and can be applied widely, but the present disclosure is not limited thereto. Specifically, the functional group 114 can be a protected amino group. Therefore, it is favorable for the applicability of the polyproline nanocage compound 100.

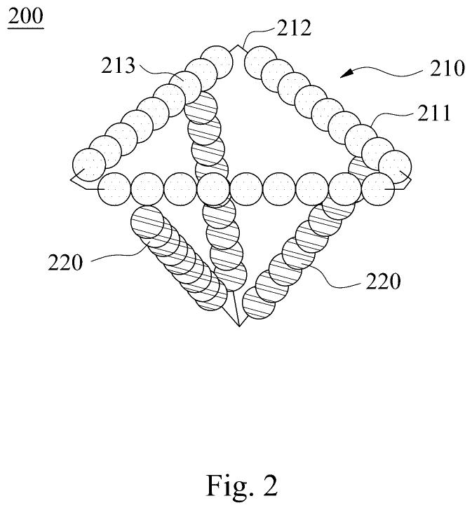

Reference is made to FIG. 2, which is a schematic view of a polyproline nanocage compound 200 according to another embodiment of the present disclosure. In FIG. 2, the polyproline nanocage compound 200 includes a cyclic peptide scaffold 210 and at least three connecting molecules 220.

In detail, the cyclic peptide scaffold 210 includes at least three polyproline helix rods 211 and at least three turn-angle molecules 212, two of the at least three polyproline helix rods 211 are connected by one of the at least three turn-angle molecules 212 so as to form a closed ring, and each of the at least three polyproline helix rods 211 is composed of a plurality of repeat units 213, and each of the repeat units 213 is represented by Formula (i):

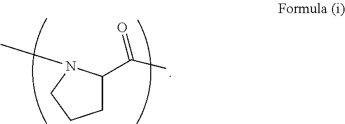

Moreover, one terminus of each of the at least three connecting molecules 220 is connected to one of the at least three polyproline helix rods 211 of the cyclic peptide scaffold 210, and the other terminus of each of the at least three connecting molecules 220 is connected to the other terminus of another of the at least three connecting molecules 220.

The detail containing of the polyproline helix rods 211, the turn-angle molecules 212 and the connecting molecules 220 in FIG. 2 can be referred to the polyproline helix rods 111, the turn-angle molecules 112 and the connecting molecules 120 in FIG. 1, and the details will not be repeated here. The difference between the polyproline nanocage compound 200 in FIG. 2 and the polyproline nanocage compound 100 in FIG. 1 is that the polyproline nanocage compound 200 has only one cyclic peptide scaffold 210, so that the polyproline nanocage compound 200 is similar to a triangular pyramid shape after connecting the cyclic peptide scaffold 210 to the connecting molecules 220.

### <Manufacturing Method of Polyproline Nanocage Compound>

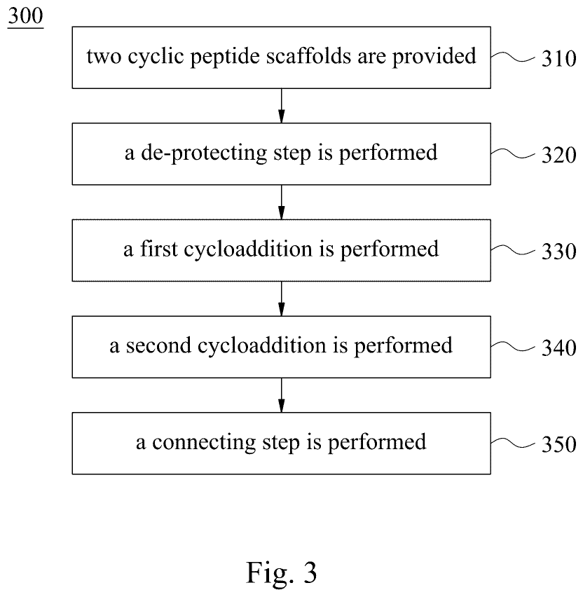

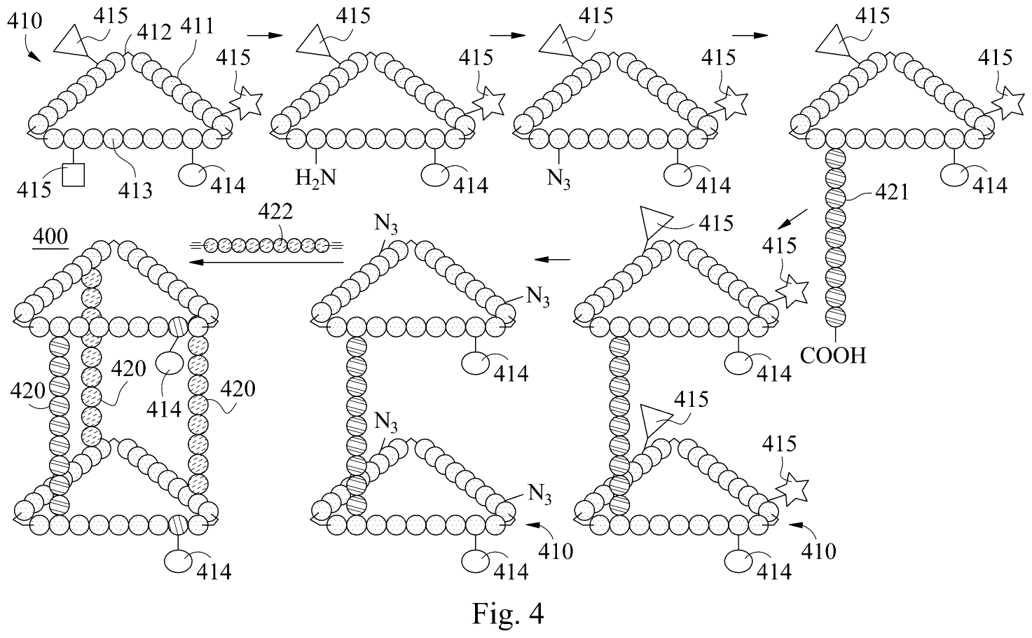

Reference is made to FIG. 3, which is a flow chart of a manufacturing method of a polyproline nanocage compound 300 according to still another embodiment of the present disclosure. In FIG. 3, the manufacturing method of the polyproline nanocage compound 300 includes step 310, step 320, step 330, step 340 and step 350. Reference is also made to FIG. 4, which is a schematic process diagram of the manufacturing method of the polyproline nanocage compound 300 according to FIG. 3. Specifically, in FIG. 4, the polyproline nanocage compound 400 is similar to a triangular prism. Therefore, the number of at least three polyproline helix rods 411, the number of at least three turn-angle molecules 412, the number of at least three modifying functional groups 415 and the number of at least three connecting molecules 420 are three in the following explanation, and so on for the rest types.

In step 310, two cyclic peptide scaffolds 410 are provided, each of the two cyclic peptide scaffolds 410 includes the at least three polyproline helix rods 411, the at least three turn-angle molecules 412 and the at least three modifying functional groups 415, two of the at least three polyproline helix rods 411 are connected by one of the at least three turn-angle molecules 412 so as to form a closed ring, and each of the at least three polyproline helix rods 411 is composed of a plurality of repeat units 413, and each of the repeat units 413 is represented by Formula (i):

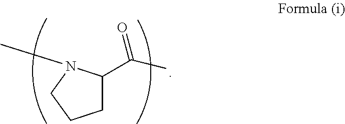

The containing of the polyproline helix rods 411 and the turn-angle molecules 412 can be referred to the above, and the details will not be repeated here. Moreover, the at least three modifying functional groups 415 are respectively connected to the at least three polyproline helix rods 411, and each of the at least three modifying functional groups 415 is formed by protecting an amino group with a protecting group, wherein the protecting group of each of the modifying functional groups 415 can be independently an allyloxycarbonyl (Alloc) protecting group, a tert-butoxycarbonyl (Boc) protecting group or a benzyloxycarbonyl (Cbz) protecting group. At least one of the repeat units 413 of at least one of the polyproline helix rods 411 of each of the cyclic peptide scaffolds 410 can be connected to a functional group 414. In FIG. 4, the type, number and position of the functional group 414 of each of the cyclic peptide scaffolds 410, and the type and position of the modifying functional groups 415 are taken as examples, but the present disclosure is not limited thereto. Specifically, the functional group 414 can be a protected amino group. Therefore, it is favorable for the applicability of the polyproline nanocage compound 400.

In Step 320, a de-protecting step is performed, wherein the protecting group of one of the at least three modifying functional groups 415 of each of the two cyclic peptide scaffolds 410 is removed to form a first primary amine, and an azide compound is added so that the first primary amine is converted into a first azido group. In detail, when each of the modifying functional groups 415 is the Alloc protecting group or the Cbz protecting group, each of the protecting group can be removed with palladium (Pd) and other reagents. When each of the modifying functional groups 415 is the Boc protecting group, the protecting group can be removed with trifluoroacetic acid (TFA). Afterwards, trifluoromethanesulfonyl azide (TfN₃) and other reagents are added to perform a diazo transfer so that the first primary amine (—NH₂) is converted into a first azido group (—N₃).

In step 330, a first cycloaddition is performed, wherein a first connecting intermediate 421 with an alkynyl group at one terminus and with a carboxyl group at the other terminus is catalyzed via a Cu ion, so that the alkynyl group of the first connecting intermediate 421 is connected to the first azido group of one of the two cyclic peptide scaffolds 410. In detail, the cycloaddition of the present disclosure is an [3+2] azide-alkyne huisgen cycloaddtion (CuAAC) reaction, wherein the [3+2] CuAAC reaction is performed with two molcules respectively having the alkynyl group and the azido group under the catalysis of monovalent copper to form a five-member ring structure. The advantages of the reaction are the high yield, the fast reaction rate, mild reaction condition, low required concentration, and the reaction will not be performed by cross-reacting with other functional groups.

In step 340, a second cycloaddition is performed, wherein the carboxyl group of the first connecting intermediate 421 is modified to form an alkynyl terminus, so that the alkynyl terminus is connected to the first azido group of the other of the two cyclic peptide scaffolds 410. In detail, the carboxyl group of the first connecting intermediate 421 can be modified to form the alkynyl terminus through the coupling reaction using propargylamine, hexafluorophosphate azabenzotriazole tetramethyl uronium (HATU) and triethylamine (TEA).

In step 350, a connecting step is performed, wherein the protecting groups of another at least two of the at least three modifying functional groups 415 of each of the two cyclic peptide scaffolds 410 are removed to respectively form at least two second primary amines, and the at least two second primary amines are converted into at least two second azido groups. Then, at least two second connecting intermediates 422 with the alkynyl groups at two termini are catalyzed via the Cu ion, so that the at least two second azido groups of each of the two cyclic peptide scaffolds 410 are connected to the alkynyl groups of the at least two second connecting intermediates 422 so as to form a polyproline nanocage compound 400. At least three first connecting molecules 420 are respectively formed from the first connecting intermediate 421 after performing the second cycloaddition and from the at least two second connecting intermediates 422 after performing the connecting step.

In FIG. 4, the first connecting intermediate 421 and each of the second connecting intermediates 422 can be a polyproline polypeptide composed of 9 repeat units represented by Formula (i). The repeat units can be connected to the functional groups, but the present disclosure is not limited thereto. Specifically, the first connecting intermediate 421 can be represented by Formula (II), and each of the second connecting intermediates 422 can be represented by Formula (III). The first connecting intermediate 421 is referenced as SEQ ID NO: 2, wherein X at residue 1 is the derivative 1 of proline. Each of the second connecting intermediates 422 is referenced as SEQ ID NO: 3, wherein X at residue 1 is the derivative 1 of proline, and X at residue 9 is the derivative 2 of proline.

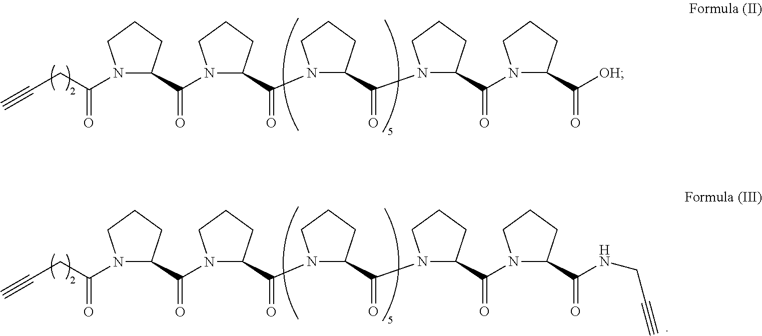

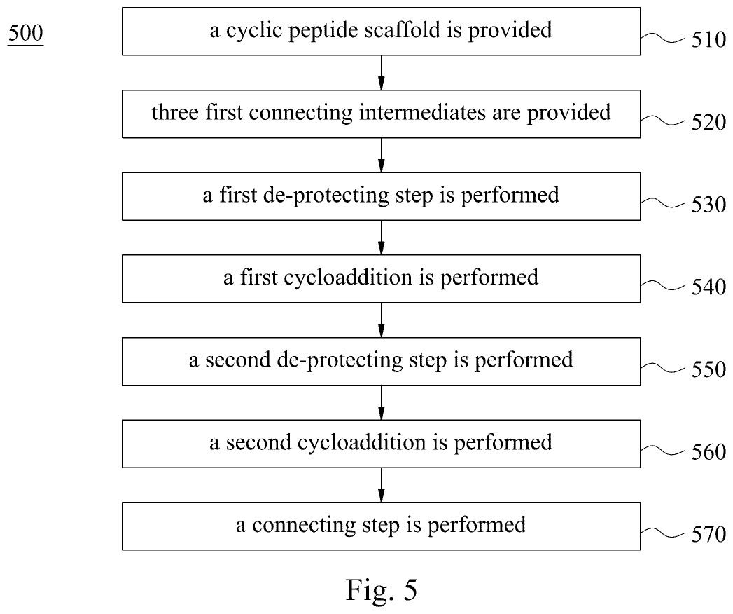

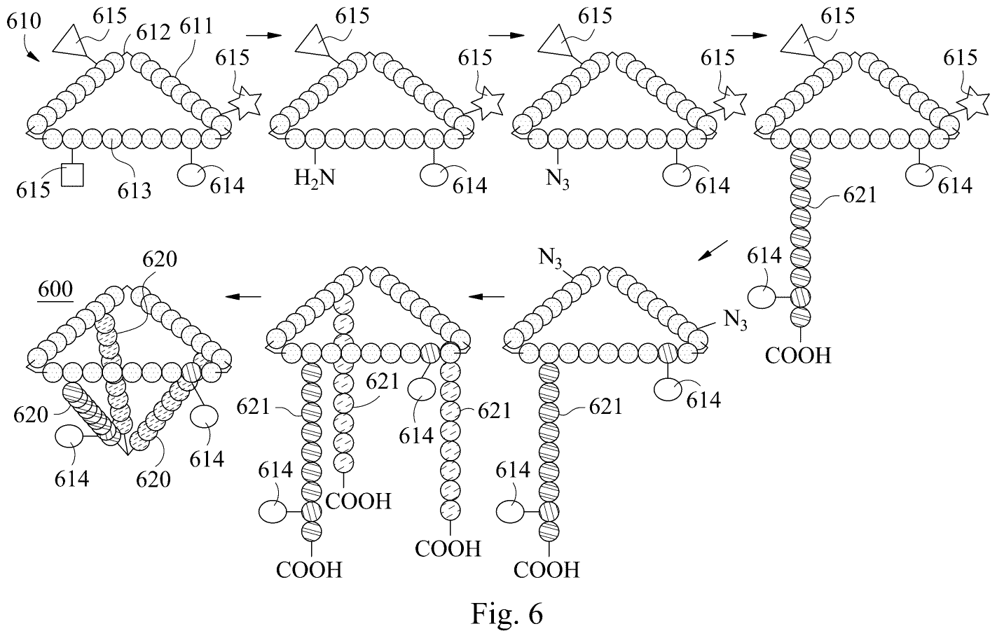

Reference is made to FIG. 5, which is a flow chart of a manufacturing method of a polyproline nanocage compound 500 according to yet another embodiment of the present disclosure. In FIG. 5, the manufacturing method of the polyproline nanocage compound 500 includes step 510, step 520, step 530, step 540, step 550, step 560 and step 570. Reference is also made to FIG. 6, which is a schematic process diagram of the manufacturing method of the polyproline nanocage compound 500 according to FIG. 5. Specifically, in FIG. 6, the polyproline nanocage compound 600 is similar to a triangular pyramid shape, so that the number of at least three polyproline helix rods 611, the number of at least three turn-angle molecules 612, the number of at least three modifying functional groups 615 and the number of at least three connecting molecules 620 are three in the following explanation, and so on for the rest types.

In step 510, a cyclic peptide scaffold 610 is provided, the cyclic peptide scaffold 610 includes the at least three polyproline helix rods 611, the at least three turn-angle molecules 612 and the at least three modifying functional groups 615, wherein two of the at least three polyproline helix rods 611 are connected by one of the at least three turn-angle molecules 612 so as to form a closed ring, and each of the at least three polyproline helix rods 611 is composed of a plurality of repeat units 613, and each of the repeat units 613 is represented by Formula (i):

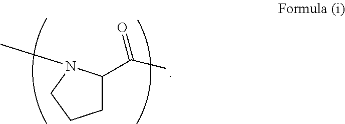

The containing of the polyproline helix rods 611 and the turn-angle molecules 612 can be referred to the above, and the details will not be repeated here. Moreover, the at least three modifying functional groups 615 are respectively connected to the at least three polyproline helix rods 611, and each of the at least three modifying functional groups 615 is formed by protecting an amino group with a protecting group, wherein the protecting group is independently an Alloc protecting group, a Boc protecting group or a Cbz protecting group. At least one of the repeat units 613 of at least one of the at least three polyproline helix rods 611 of the cyclic peptide scaffold 610 can be connected to a functional group 614. In FIG. 6, type, the number and position of the functional group 614 of the cyclic peptide scaffold 610 and the type and position of the modifying functional groups 615 are taken as examples, but the present disclosure is not limited thereto. Specifically, the functional group 614 can be a protected amino group. Therefore, it is favorable for the applicability of the polyproline nanocage compound 600.

In step 520, at least three first connecting intermediates 621 are provided, wherein each of the at least three first connecting intermediates 621 is with an alkynyl group at one terminus and with a carboxyl group at the other terminus. The detail containing of the first connecting intermediates 621 can be referred to the above, and the details will not be repeated here.

In step 530, a first de-protecting step is performed, wherein the protecting group of one of the at least three modifying functional groups 615 is removed to form a first primary amine, and an azide compound is added so that the first primary amine is converted into a first azido group. The detail containing of the first de-protecting step can be referred to the above, and the details will not be repeated here.

In step 540, a first cycloaddition is performed, wherein one of the at least three first connecting intermediates 621 is catalyzed via a Cu ion, so that the alkynyl group of the one of the at least three first connecting intermediates 621 is connected to the first azido group of the cyclic peptide scaffold 610. In FIG. 6, the repeat units of the first connecting intermediates 621 can be also connected to the functional group 614, but the present disclosure is not limited thereto. The detail containing of the first cycloaddition can be referred to the above, and the details will not be repeated here.

In step 550, a second de-protecting step is performed, wherein the protecting groups of another at least two of the at least three modifying functional groups 615 of the cyclic peptide scaffold 610 are removed to form at least two second primary amines, so that the at least two second primary amines are converted into at least two second azido groups.

In step 560, a second cycloaddition is performed, wherein another at least two of the at least three first connecting intermediates 621 are catalyzed via the Cu ion, so that the alkynyl groups of the another at least two of the at least three first connecting intermediates 621 are connected to the at least two second azido groups of the cyclic peptide scaffold 610.

In step 570, a connecting step is performed, the carboxyl groups of the at least three first connecting intermediates 621 are modified to azido groups, and are connected to each other so as to form a polyproline nanocage compound 600, wherein the at least three connecting molecules 620 are formed from the at least three first connecting intermediates 621 after performing the connecting step. In detail, the carboxyl groups of the first connecting intermediates 621 are modified to azido groups with 3-azido-1-propylamine, HATU and TEA. Afterwards, a molecule with three alkynyl groups is connected to the azido groups of the first connecting intermediates 621 so that the first connecting intermediates 621 are connected to each other.

Moreover, it should be noted that the above-mentioned first primary amines and second primary amines, and first azido groups and second azido groups are used to clearly indicate the order of combination, and their essences are the same functional groups.

### <Use of Polyproline Nanocage Compound>

The present disclosure provides a use of a polyproline nanocage compound, which can be used as an artificial enzyme or an artificial receptor. In detail, the polyproline nanocage compound of the present disclosure has a cavity so as to host various guest molecules for reactions. Moreover, the polyproline nanocage compound is a biocompatible molecule with stable structure in the aqueous solution, and has characteristics of rigidity and molecular ruler so that the polyproline helix rods can be modified with specific functional groups or specific molecular fragments. Therefore, the location of multiple functional groups or molecular fragments can be precisely adjusted by changing the modified positions of the polyproline in a three-dimensional space (cavity) so as to efficiently catalyze reactions, or recognize enzyme substrates or biological ligand molecules. Therefore, the polyproline nanocage compound composed of the polyproline helix rods can selectively interact with the target molecules and is suitable as the artificial enzyme or the artificial receptor.

The following specific embodiments further illustrate the present disclosure for those with ordinary skill in the technical field to utilize and realize the present disclosure without excessive interpretation. These embodiments should not limit the scope of the present disclosure, but illustrate how to implement the materials and methods of the present disclosure.

In the present disclosure, in order to connect two cyclic peptide scaffolds to each other, modifying functional groups for the subsequent reactions should be connected to the cyclic peptide scaffolds, and the modifying functional groups must avoid effects on the structure of the polyproline helix rods. Specifically, proline building units of Synthesis example 1 to Synthesis example 4 of the present disclosure are used to perform the synthesis of the polyproline helix rods, and the structures of the proline building units of Synthesis example 1 to Synthesis example 4 are shown in Table 1.

TABLE 1
Synthesis example 1

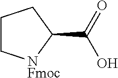

Synthesis example 2

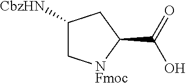

Synthesis example 3

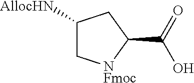

Synthesis example 4

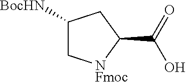

### <Synthesis of Polyproline Helix Rod>

Step (1): Synthesis example 1 and N-Methylmorpholine (NMM) are dissolved in a solution of dichloromethane (DCM) and dimethyl formamide (DMF) in a ratio of 1:1, 2-chlorotrityl chloride resin is added into the solution, and then a solution of methanol, dichloromethane and diisopropylethylamine (DIEA) in a ratio of 2:17:1 is added and shaken evenly.

Step (2): 2-chlorotrityl chloride resin treated in step (1) is soaked in DMF with 10% piperidine to react for 10 minutes, so that the Fmoc protecting groups on Synthesis example 1 are removed, and the reactive amine groups at the peptide N-termini are obtained.

Step (3): proline building units of Synthesis example 1 to Synthesis example 4 as reactants are mixed with a coupling agent of Benzotriazol-1-yl-oxytripyrrolidinophosphonium hexafluorophosphate (PyBOP) or HATU, and are dissolved in DMF with adding NMM so as to complete the preparation of the reaction solution. Moreover, the reaction solution is mixed with the resin for 1 hour to complete the coupling reaction. Since the effect of the modifying functional groups on the side chains of the proline building units of Synthesis example 2 to Synthesis example 4 may cause the coupling difficulties, so that HATU is used to enhance the coupling efficiency.

Step (4): the resin is reacted with a de-active reagent of acetic anhydride (Ac₂O) and pyridine in a ratio of 1:9 for 10 minutes, Step (2) to Step (4) are repeated 8 times, and then product A is obtained. The proline building unit can be replaced as needed during repeating Step (3), so that types and linked positions of the modifying functional groups can be controlled.

Step (5): product A is mixed with a reaction reagent of DCM, triisopropylsilane (TIS) and TFA in a ratio of 90:5:5 to react for 1 hour, the peptides and the resin are separated, and the obtained mixed solution is purified with a high performance liquid chromatography and a freeze-dried process to remove the solvents, so that the polyproline helix rods with carboxyl groups at the C-termini are obtained.

The reaction equation of the polyproline helix rods of the present disclosure is shown in Table 2.

TABLE 2

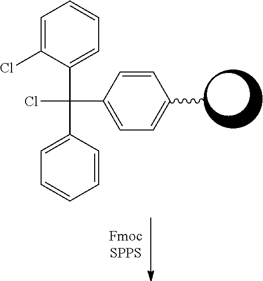

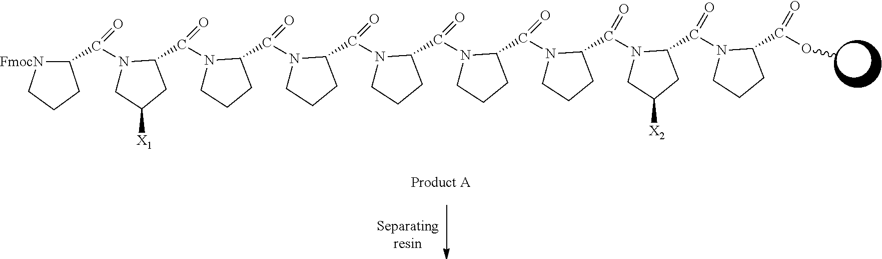

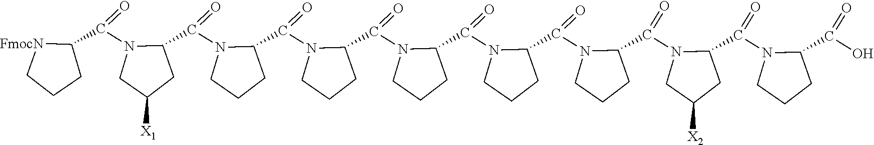

Moreover, in order to perform the synthesis of the cyclic peptide scaffold, the polyproline helix rods are mixed with a solution of propargylamine, HATU and TEA and dissolved in DMF to react, and purified with the high performance liquid chromatography and the freeze-dried process to remove the solvents so as to obtain product B with the carboxyl groups modified to the alkynyl groups. The reaction equation is shown in Table 3.

TABLE 3

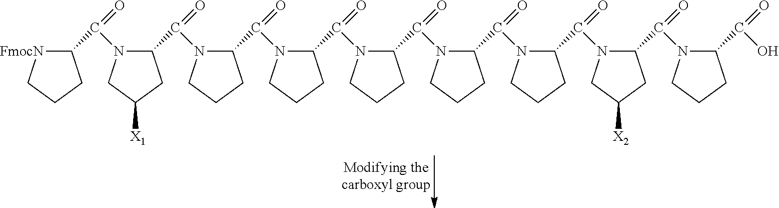

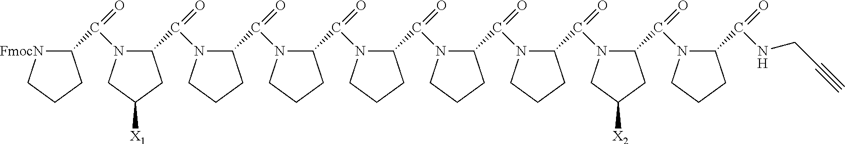

Product B

According to the above, X₁ and X₂ of the polyproline helix rods of Synthesis example 5 to Synthesis example 10 synthesized by the proline building units of Synthesis example 1 to Synthesis example 4 are shown in Table 4. The polyproline helix rods of Synthesis example 5 is referenced as SEQ ID NO: 4, wherein X at residue 1 is the derivative 3 of proline, X at residue 8 is the derivative 4 of proline, and X at residue 9 is the derivative 2 of proline. The polyproline helix rods of Synthesis example 6 is referenced as SEQ ID NO: 5, wherein X at residue 1 is the derivative 3 of proline, X at residue 2 is the derivative 5 of proline, X at residue 8 is the derivative 4 of proline, and X at residue 9 is the derivative 2 of proline. The polyproline helix rods of Synthesis example 7 is referenced as SEQ ID NO: 6, wherein X at residue 1 is the derivative 3 of proline, X at residue 8 is the derivative 6 of proline, and X at residue 9 is the derivative 2 of proline. The polyproline helix rods of Synthesis example 8 is referenced as SEQ ID NO: 7, wherein X at residue 1 is the derivative 3 of proline, X at residue 2 is the derivative 5 of proline, X at residue 8 is the derivative 6 of proline, and X at residue 9 is the derivative 2 of proline. The polyproline helix rods of Synthesis example 9 is referenced as SEQ ID NO: 8, wherein X at residue 1 is the derivative 3 of proline, X at residue 2 is the derivative 6 of proline, X at residue 8 is the derivative 5 of proline, and X at residue 9 is the derivative 2 of proline. The polyproline helix rods of Synthesis example 10 is referenced as SEQ ID NO: 9, wherein X at residue 1 is the derivative 3 of proline, X at residue 2 is the derivative 4 of proline, and X at residue 9 is the derivative 2 of proline.

TABLE 4

| | X₁ | X₂ |
|---|---|---|
| Synthesis example 5 | H | NHAlloc |
| Synthesis example 6 | NHCbz | NHAlloc |
| Synthesis example 7 | H | NHBoc |
| Synthesis example 8 | NHCbz | NHBoc |
| Synthesis example 9 | NHBoc | NHCbz |
| Synthesis example 10 | NHAlloc | H |

### <Synthesis of Cyclic Peptide Scaffold>

Step (1): product A is soaked in DMF with 10% piperidine to react for 10 minutes, so that the Fmoc protecting groups on the residue are removed, and the reactive terminal amine groups are obtained.

Step (2): molecule S with azido group as reactant are dissolved in DMF with adding DIEA, and reacts with product A for 1 hour to complete the coupling reaction. Product A′ with azido group at terminus is formed through the coupling reaction that molecule S is connected to the N-terminus of product A, wherein the carboxyl groups of molecule S is activated into Osu ester, and there is no need to add a coupling reagent.

Step (3): product A′ from step (2) and product B as reactants, to perform CuAAC reaction in tetrahydrofuran (THF), so that a cycloaddition is performed with the azido group of product A′ and the alkynyl group of product B to connect product A′ and product B. When the cycloaddition is completed, the Cu ions are removed after being washed by the sodium diethyldithiocarbamate solution in DMF. Afterwards, the steps of removing the Fmoc protecting groups and connecting the molecule S are repeated, and the CuAAC reaction is performed with product B based on step (3). Furthermore, products B used in sequence are product B-1 and product B-2, respectively, and after the cycloaddition, removing the Fmoc protecting groups and connecting the molecule S are repeated so as to obtain product C.

Step (4): product C is mixed with a reaction reagent of DCM, TIS and TFA in a ratio of 96:2:2 to react for 1 hour, the peptides and the resin are separated, and the obtained mixed solution is purified with a high performance liquid chromatography and a freeze-dried process to remove the solvents, so that product D with the carboxyl group at the C-terminus is obtained.

Step (5): product D is mixed with a solution of propargylamine, HATU and TEA and dissolved in DMF to react, and purified with the high performance liquid chromatography and the freeze-dried process to remove the solvents so as to obtain product E with carboxyl groups modified to alkynyl groups.

Step (6): cyclic peptide scaffolds of Synthesis example 11, Synthesis example 12 and Synthesis example 13 are obtained by the CuAAC reaction with a self-cyclization reaction of product E.

According to the above, the structures of product A′, molecule S, product B-1, product B-2, product C, product D, product E and cyclic peptide scaffold are shown in Table 5.

TABLE 5
product A′

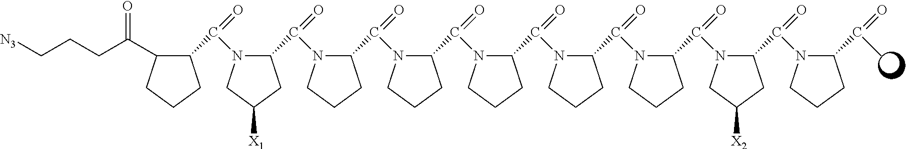

molecule S

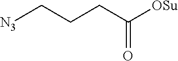

product B-1

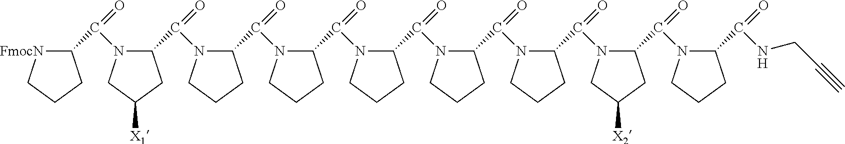

product B-2

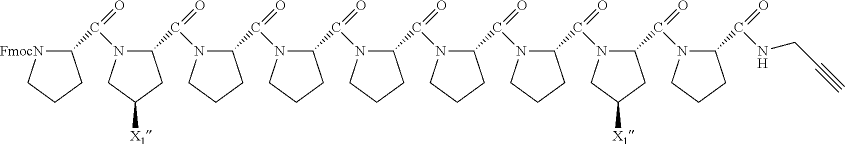

product C

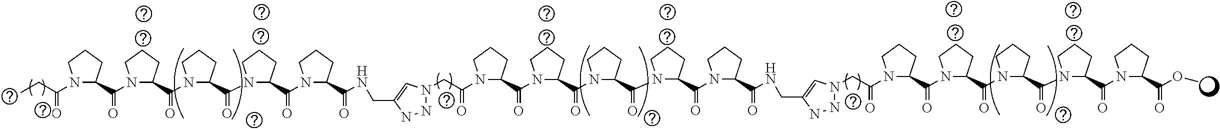

product D

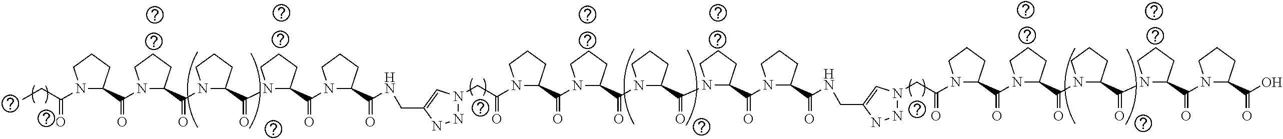

product E

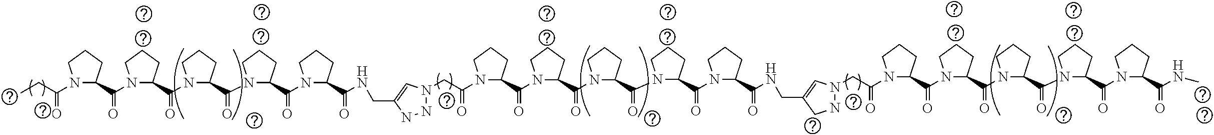

cyclic peptide scaffold

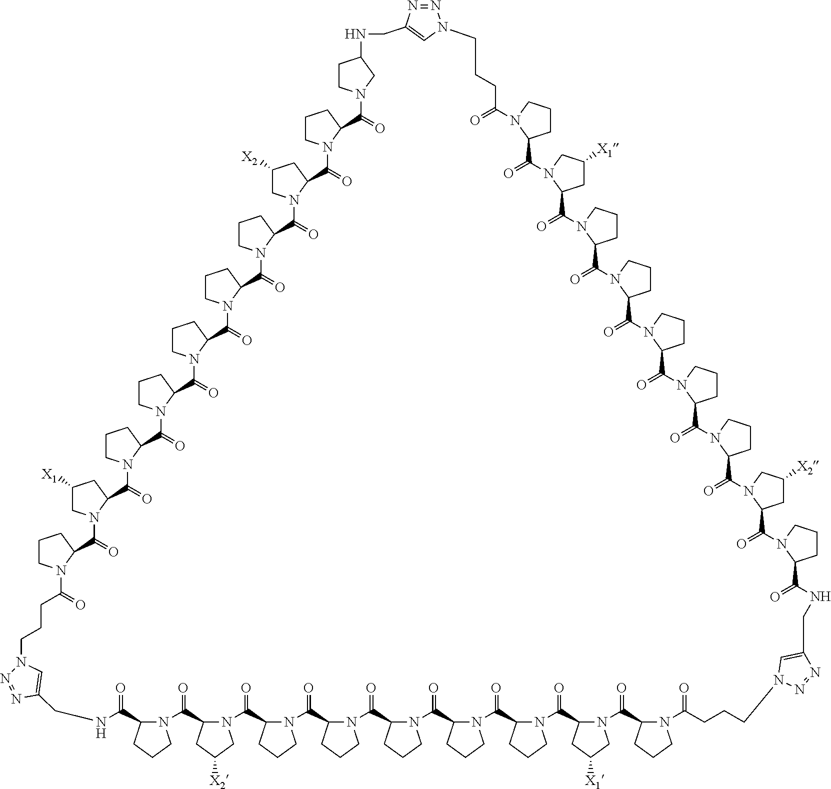

Specifically, the parameters of each of the cyclic peptide scaffolds of Synthesis example 11, each of the cyclic peptide scaffolds of Synthesis example 12 and each of the cyclic peptide scaffolds of Synthesis example 13 are shown in Table 6. Each of the cyclic peptide scaffolds of Synthesis example 11 is referenced as SEQ ID NO: 10, wherein X at residue 8 is the derivative 6 of proline, X at residue 17 is the derivative 4 of proline, X at residue 20 is the derivative 5 of proline, and X at residue 26 is the derivative 4 of proline. Each of the cyclic peptide scaffolds of Synthesis example 12 is referenced as SEQ ID NO: 11, wherein X at residue 2 is the derivative 5 of proline, X at residue 8 is the derivative 6 of proline, X at residue 17 is the derivative 4 of proline, and X at residue 26 is the derivative 4 of proline. Each of the cyclic peptide scaffolds of Synthesis example 13 is referenced as SEQ ID NO: 12, wherein X at residue 2 is the derivative 6 of proline, X at residue 8 is the derivative 5 of proline, X at residue 11 is the derivative 4 of proline, and X at residue 20 is the derivative 4 of proline.

TABLE 6

| | Synthesis | Synthesis | Synthesis |
|---|---|---|---|
| | example 11 | example 12 | example 13 |
| X₁ | H | NHCbz | NHBoc |
| X₂ | NHBoc | NHBoc | NHCbz |
| X₁′ | NHCbz | H | NHAlloc |
| X₂′ | NHAlloc | NHAlloc | H |
| X₁″ | H | H | NHAlloc |
| X₂″ | NHAlloc | NHAlloc | H |

### <Synthesis of Polyproline Nanocage Compound>

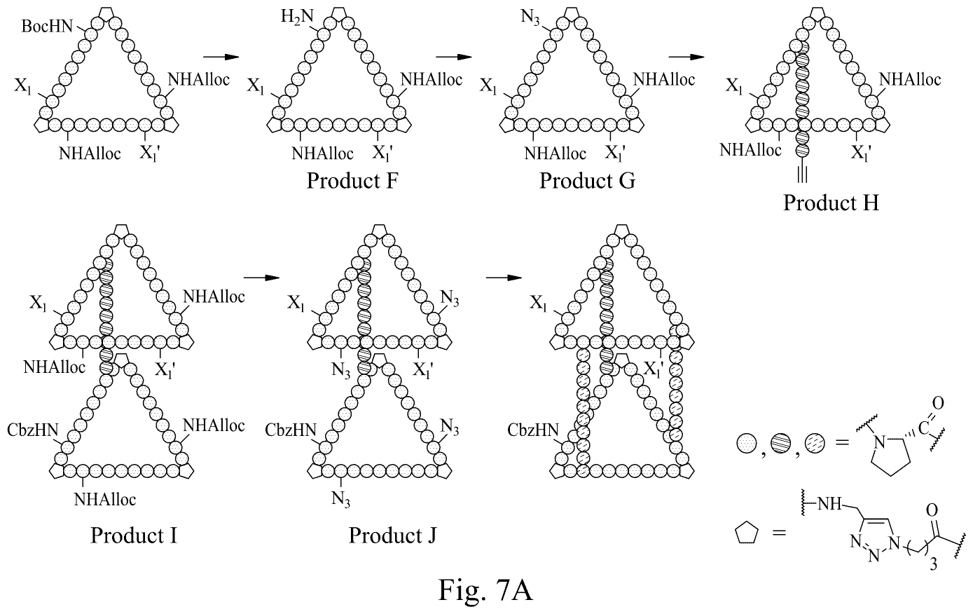

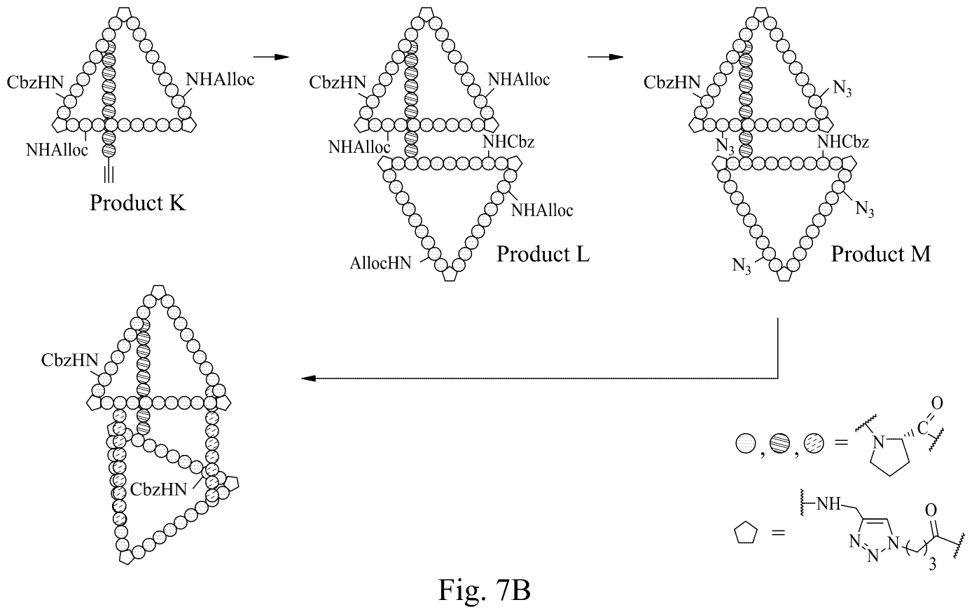

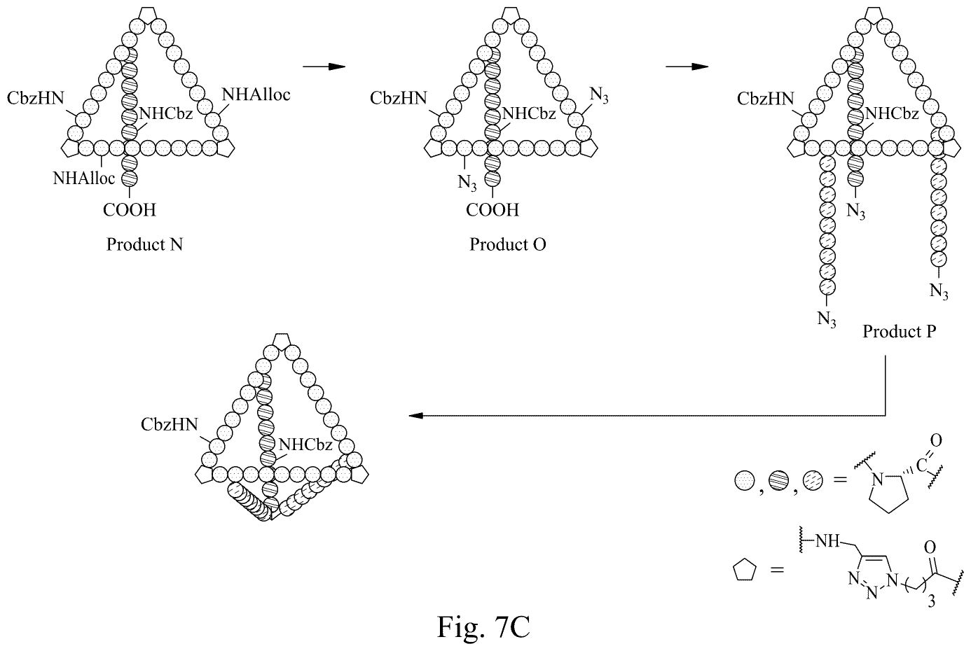

In order to clearly and succinct present the manufacturing process and the structure of the polyproline nanocage compound, the present disclosure is illustrated by diagrams. Reference is made to FIG. 7A, FIG. 7B and FIG. 7C, wherein FIG. 7A is a manufacturing process diagram of a polyproline nanocage compound according to Example 1 and Example 2; FIG. 7B is a manufacturing process diagram of a polyproline nanocage compound according to Example 3, and FIG. 7C is a manufacturing process diagram of a polyproline nanocage compound according to Example 4.

In FIG. 7A, Boc protecting groups of Synthesis example 11 or Synthesis example 12 are removed by TFA to obtain product F of a primary amine. TfN₃, copper sulfate and TEA are added, and a diazo transferring reaction is performed to convert the primary amine into an azido group so as to obtain product G. Afterwards, the CuAAC reaction is performed by a first connecting intermediate represented by Formula (II) reacting with product G, then the resulting product is mixed with a solution of propargylamine, HATU and TEA, and dissolved in DMF so as to obtain product H with a carboxyl at C-terminus modified to the alkynyl group. Then, Boc protecting group of Synthesis example 13 is removed, a diazo transferring reaction is performed, and the CuAAC reaction is performed with product H so as to obtain product I through connecting two cyclic peptide scaffolds. Furthermore, primary amines are obtained by removing rest Alloc protecting groups, and a diazo transferring reaction is performed to convert the primary amines into azido groups so as to obtain product J. Finally, the CuAAC reaction is performed by two second connecting intermediates represented by Formula (III) reacting with product J so as to obtain nanocage compounds of Example 1 and Example 2. In FIG. 7A, X₁ and X₂ of the nanocage compounds of Example 1 and Example 2 are shown in Table 7.

TABLE 7

| | Example 1 | Example 2 |
|---|---|---|
| X₁ | H | NHCbz |
| X₁′ | NHCbz | H |

Specifically, take Example 2 as the example, the polyproline nanocage compound with a triangular prism shape is shown in Table 8. Each of the connecting molecules of the polyproline nanocage compound according to Example 1 is referenced as SEQ ID NO: 1. One of the cyclic peptide scaffolds of the polyproline nanocage compound according to Example 1 is referenced as SEQ ID NO: 13, wherein X at residue 20 is the derivative 5 of proline, and prolines at residue 8, 17 and 26 are respectively connected to the connecting molecules. The other of the cyclic peptide scaffolds of the polyproline nanocage compound according to Example 1 is referenced as SEQ ID NO: 14, wherein X at residue 8 is the derivative 5 of proline, and prolines at residue 2, 11 and 20 are respectively connected to the connecting molecules. Each of the connecting molecules of the polyproline nanocage compound according to Example 2 is referenced as SEQ ID NO: 1. One of the cyclic peptide scaffolds of the polyproline nanocage compound according to Example 2 is referenced as SEQ ID NO: 14, wherein X at residue 8 is the derivative 5 of proline, and prolines at residue 2, 11 and 20 are respectively connected to the connecting molecules. The other of the cyclic peptide scaffolds of the polyproline nanocage compound according to Example 2 is referenced as SEQ ID NO: 15, wherein X at residue 2 is the derivative 5 of proline, and prolines at residue 8, 17 and 26 are respectively connected to the connecting molecules.

TABLE 8

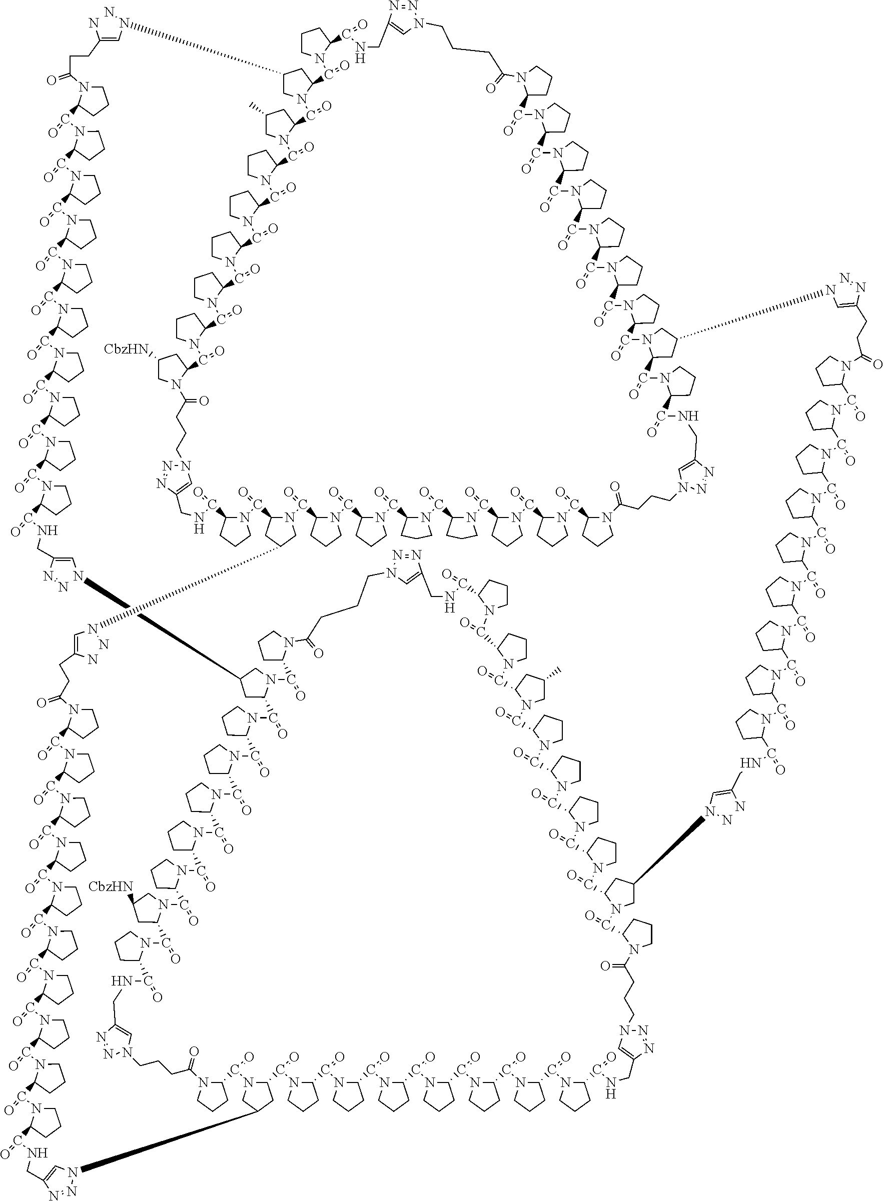

In FIG. 7B, Boc protecting group of Synthesis example 12 is removed, a diazo transferring reaction is performed, the CuAAC reaction is performed with a first connecting intermediate represented by Formula (II), mixed with a solution of propargylamine, HATU and TEA, and dissolved in DMF so as to obtain product K with the carboxyl terminus modified to the alkynyl group. Then, Boc protecting group of the cyclic peptide scaffold of Synthesis example 12 is removed, a diazo transferring reaction is performed, and the CuAAC reaction is performed with product K so as to obtain product L through connecting two cyclic peptide scaffolds. Furthermore, primary amines are obtained by removing rest Alloc protecting groups, and a diazo transferring reaction is performed to convert the primary amines into azido groups so as to obtain product M. Finally, the CuAAC reaction is performed by two second connecting intermediates represented by Formula (III) reacting with product M so as to obtain polyproline nanocage compound of Example 3. Each of the connecting molecules of the polyproline nanocage compound according to Example 3 is referenced as SEQ ID NO: 1. Each of the cyclic peptide scaffolds of the polyproline nanocage compound according to Example 3 is referenced as SEQ ID NO: 15, wherein X at residue 2 is the derivative 5 of proline, and prolines at residue 8, 17 and 26 are respectively connected to the connecting molecules.

In FIG. 7C, Boc protecting group of cyclic peptide scaffold of Synthesis example 13 is removed, a diazo transferring reaction is performed, and the CuAAC reaction is performed with a first connecting intermediates represented by Formula (II-1) so as to obtain product N. Furthermore, primary amines are obtained by removing rest Alloc protecting groups, and a diazo transferring reaction is performed to convert the primary amines into azido groups so as to obtain product O. The CuAAC reaction is performed with the first connecting intermediate represented by Formula (II) and product O, and mixed with a solution of 3-azido-1-propylamine, HATU and TEA, and dissolved in DMF so as to obtain product P by modifying the carboxyl terminus of the first connecting intermediate to the azido group. Finally, three first connecting intermediates are connected to a molecule with three alkynyl groups represented by Formula (IV) so that the three first connecting intermediates are connected to each other so as to obtain a polyproline nanocage compound of Example 4. Furthermore, in other example, the first connecting intermediate may be but not be limited to the structures represented by Formula (II), Formula (II-1) or Formula (II-2). For example, Example 5 of the present disclosure can react with the first connecting intermediate represented by Formula (II) via the CuAAC reaction, then reacted with the first connecting intermediate represented by Formula (II-2) via the CuAAC reaction, and the remaining steps are the same as the steps in Example 4 so as to obtain the polyproline nanocage compound.

The first connecting intermediates represented by Formula (II-1) and Formula (II-2) and a molecule with three alkynyl groups represented by Formula (IV) are shown in Table 9. The first connecting intermediate represented by Formula (II-1) is referenced as SEQ ID NO: 16, wherein X at residue 1 is the derivative 1 of proline, and X at residue 6 is the derivative 5 of proline. The first connecting intermediate represented by Formula (II-2) is referenced as SEQ ID NO: 17, wherein X at residue 1 is the derivative 1 of proline, and X at residue 2 is the derivative 4 of proline.

TABLE 9

| | |
|---|---|
| 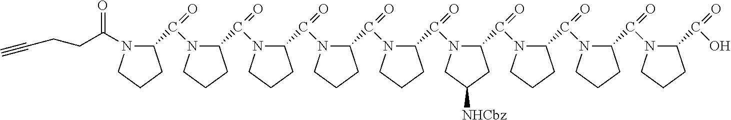 [SMILES: C#CCCC(=O)N1CCC[C@H]1C(=O)N1CCC[C@H]1C(=O)N1CCC[C@H]1C(=O)N1CCC[C@H]1C(=O)N1CCC[C@H]1C(=O)N1C[C@H](NC(=O)OCc2ccccc2)C[C@H]1C(=O)N1CCC[C@H]1C(=O)N1CCC[C@H]1C(=O)N1CCC[C@H]1C(=O)O] | For- mula (II-1) |
| 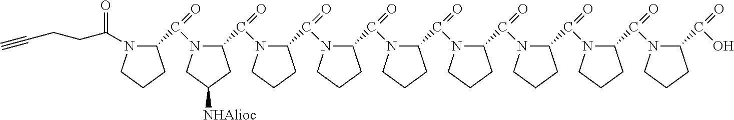 | For- mula (II-2) |
| 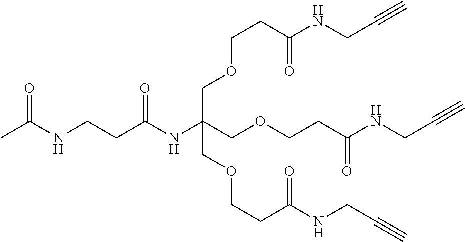 [SMILES: C#CCNC(=O)CCOCC(COCCC(=O)NCC#C)(COCCC(=O)NCC#C)NC(=O)CCNC(C)=O] | For- mula (IV) |

Specifically, take Example 4 as an example, the polyproline nanocage compound with a triangular pyramid shape is shown in Table 10. Two of the connecting molecules of the polyproline nanocage compound according to Example 4 are respectively referenced as SEQ ID NO: 1, and the other of the connecting molecules of the polyproline nanocage compound according to Example 4 is referenced as SEQ ID NO: 16. The cyclic peptide scaffold of the polyproline nanocage compound according to Example 4 is referenced as SEQ ID NO: 14, wherein X at residue 8 is the derivative 5 of proline, and prolines at residue 2, 11 and 20 are respectively connected to the connecting molecules.

TABLE 10

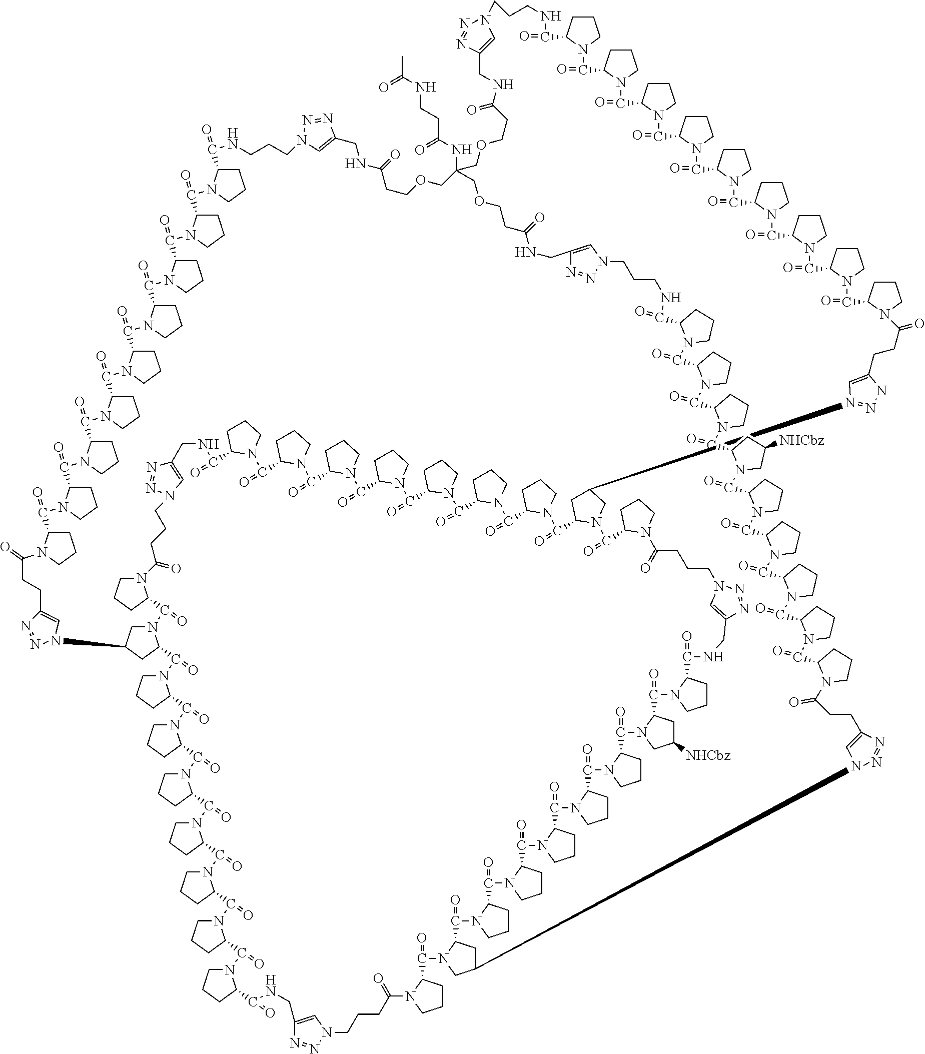

### <Use of Polyproline Nanocage Compound>

Polyproline nanocage compounds of Example 6 to Example 11 are synthesized from the cyclic peptide scaffolds of Synthesis example 14 and Synthesis example 15, phenylboronic acid side chains with different lengths are connected to the cyclic peptide scaffolds, and the selectivity to cyclodextrins with different sizes are achieved by changing the location or the length of the phenylboronic acid. Moreover, the interaction between the cyclic peptide scaffolds modified by phenylboronic acid and the cyclodextrins is determined by the surface plasmon resonance analysis, so that it can illustrate that the polyproline nanocage compounds of the present disclosure can be used as an artificial receptor.

A manufacturing method of the cyclic peptide scaffold of Synthesis example 14 and Synthesis example 15 is the same as the manufacturing method of the cyclic peptide scaffold of Synthesis example 11 to Synthesis example 13, and the difference is types and numbers of the proline building units. X¹ to X³ and the structures of the cyclic peptide scaffolds of Synthesis example 14 and Synthesis example 15 are shown in Table 11. The cyclic peptide scaffold of Synthesis example 14 is referenced as SEQ ID NO: 18, wherein X at residue 2 is the derivative 5 of proline, X at residue 8 is the derivative 5 of proline, X at residue 14 is the derivative 5 of proline, and X at residue 16 is the derivative 4 of proline. The cyclic peptide scaffold of Synthesis example 15 is referenced as SEQ ID NO: 19, wherein X at residue 3 is the derivative 5 of proline, X at residue 9 is the derivative 5 of proline, X at residue 15 is the derivative 5 of proline, and X at residue 16 is the derivative 4 of proline.

TABLE 11

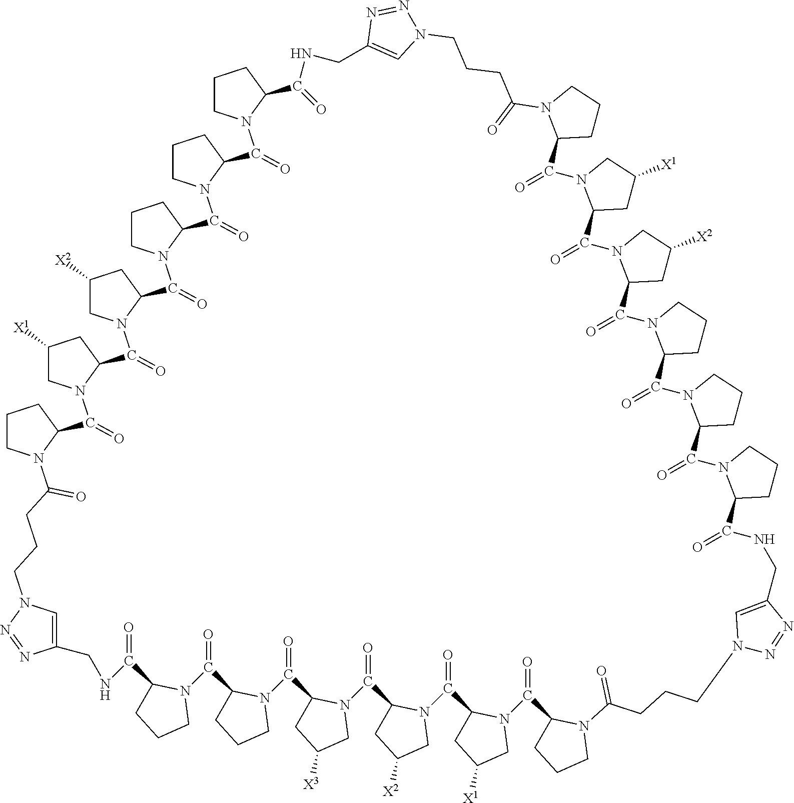

| | Synthesis example 14 | Synthesis example 15 |
|---|---|---|
| X¹ | NHCbz | H |
| X² | H | NHCbz |
| X³ | NHAlloc | NHAlloc |

Furthermore, Alloc protecting groups of Synthesis example 14 and Synthesis example 15 are removed to form amino group (NH₂), a coupling reaction is performed with amino group connecting molecules by HATU and TEA, and the cyclic peptide scaffolds with Boc protecting groups at termini can be obtained. Moreover, Cbz protecting groups of Synthesis example 14 and Synthesis example 15 are removed, coupling reaction is performed with azido group connecting molecules by HATU, the azido groups of Synthesis example 14 and Synthesis example 15 are connected to the alkyne group of the boronic acid connecting molecules through the CuAAC, and Boc protecting group is removed to form amino group at the terminus. Finally, the polyproline nanocage compounds of Example 6 to Example 11 synthesized from the cyclic peptide scaffolds can be an artificial receptor. The structures of the amino group connecting molecule, the azido group connecting molecule and the boronic acid connecting molecule are shown in Table 12.

TABLE 12

| | |
|---|---|
| amino group connecting molecule | 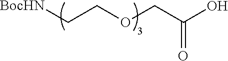 |
| azido group connecting molecule | 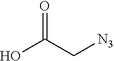 |
| boronic acid connecting molecule | 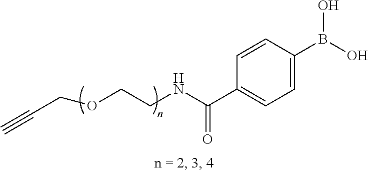 |

The structures of each of the cyclic peptide scaffolds of the polyproline nanocage compounds of Example 6 to Example 8 are shown in Table 13. Each of the cyclic peptide scaffolds of the polyproline nanocage compounds of Example 6 is referenced as SEQ ID NO: 20, wherein X at residue 2 is the derivative 7 of proline, X at residue 8 is the derivative 7 of proline, X at residue 14 is the derivative 7 of proline, and X at residue 16 is the derivative 10 of proline. Each of the cyclic peptide scaffolds of the polyproline nanocage compounds of Example 7 is referenced as SEQ ID NO: 21, wherein X at residue 2 is the derivative 8 of proline, X at residue 8 is the derivative 8 of proline, X at residue 14 is the derivative 8 of proline, and X at residue 16 is the derivative 10 of proline. Each of the cyclic peptide scaffolds of the polyproline nanocage compounds of Example 8 is referenced as SEQ ID NO: 22, wherein X at residue 2 is the derivative 9 of proline, X at residue 8 is the derivative 9 of proline, X at residue 14 is the derivative 9 of proline, and X at residue 16 is the derivative 10 of proline.

TABLE 13

| | |
|---|---|
| struc- ture | 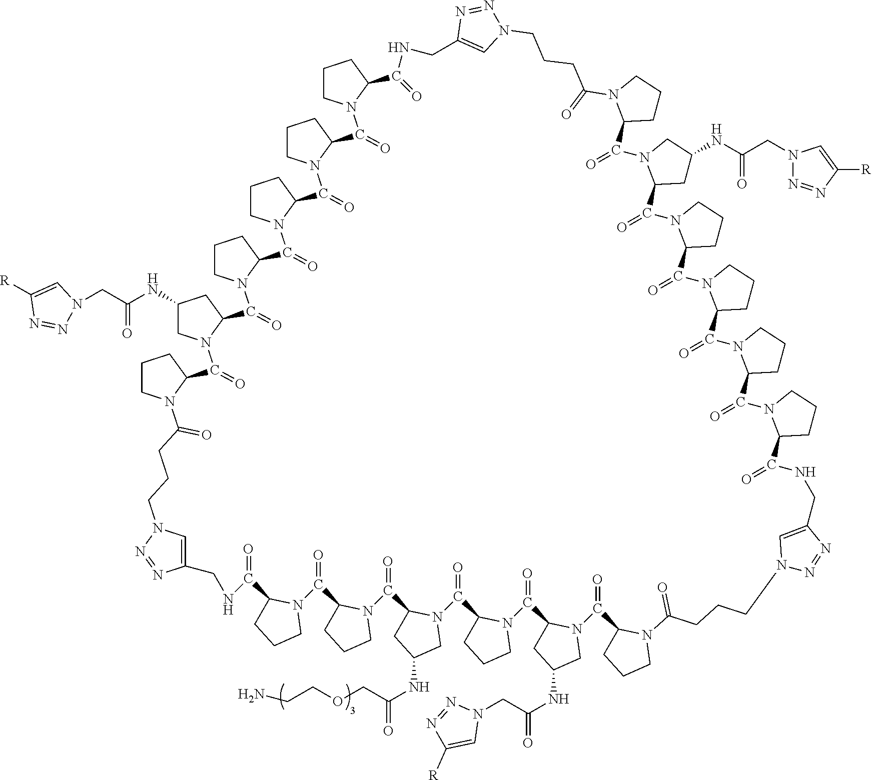 |
| Ex- am- ple 6 | 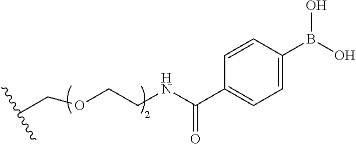 |
| Ex- am- ple 7 | 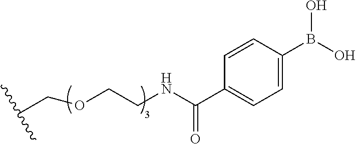 |
| Ex- am- ple 8 | 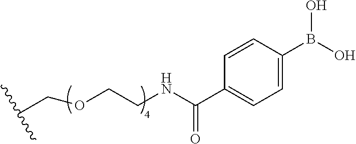 |

The structures of each of the cyclic peptide scaffolds of the polyproline nanocage compounds of Example 9 to Example 11 are shown in Table 14. Each of the cyclic peptide scaffolds of the polyproline nanocage compounds of Example 9 is referenced as SEQ ID NO: 23, wherein X at residue 3 is the derivative 7 of proline, X at residue 9 is the derivative 7 of proline, X at residue 15 is the derivative 7 of proline, and X at residue 16 is the derivative 10 of proline. Each of the cyclic peptide scaffolds of the polyproline nanocage compounds of Example 10 is referenced as SEQ ID NO: 24, wherein X at residue 3 is the derivative 8 of proline, X at residue 9 is the derivative 8 of proline, X at residue 15 is the derivative 8 of proline, and X at residue 16 is the derivative 10 of proline. Each of the cyclic peptide scaffolds of the polyproline nanocage compounds of Example 11 is referenced as SEQ ID NO: 25, wherein X at residue 3 is the derivative 9 of proline, X at residue 9 is the derivative 9 of proline, X at residue 15 is the derivative 9 of proline, and X at residue 16 is the derivative 10 of proline.

TABLE 14

| | |
|---|---|
| struc- ture | 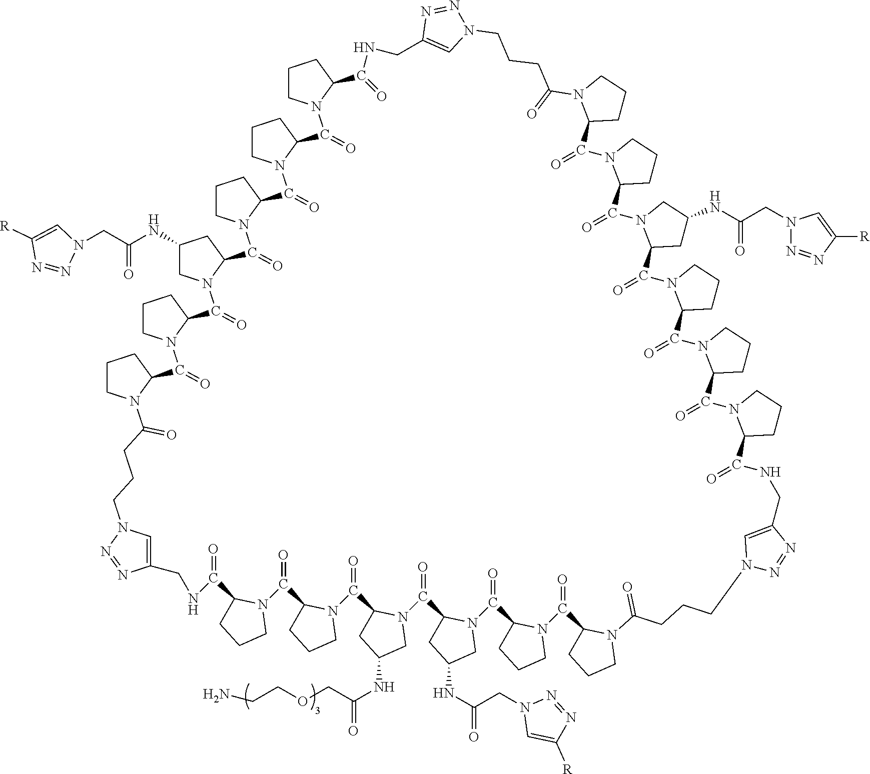 |
| Ex- am- ple 9 | 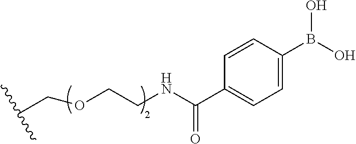 |
| Ex- am- ple 10 | 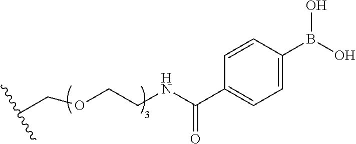 |
| Ex- am- ple 11 | 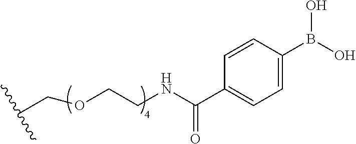 |

In order to measure the interaction of the polyproline nanocage compound with the cyclodextrins, the surface plasmon resonance analysis is used to measure the dissociation constant (K_{d}) of the interaction. Firstly, the amino groups of Example 6 and Example 8 are connected to the surface of a chip via the coupling reaction, and a kinetic testing of their interaction to α-cyclodextrin (α-CD), β-cyclodextrin (β-CD) and γ-cyclodextrin (γ-CD) is performed on the chip to obtain the K_{d} values shown in Table 15.

TABLE 15

| | α-CD | β-CD | γ-CD |
|---|---|---|---|
| Example 6 | 684.6 ± 85.9 | 86.25 ± 29.3 | 871.2 ± 134.9 |
| Example 8 | 257 ± 18.2 | 35.3 ± 1.2 | 201 ± 102.2 |

In Table 15, the interaction of Example 6 to β-CD is better than to α-CD and γ-CD, the same trend is also observed in Example 8, which has a better interaction than Example 6 to the three cyclodextrins. The reason for the better interaction of Example 8 is that the side chain of the boronic acid is longer and easily collides with the cyclodextrins. Therefore, the interaction of the cyclic peptide scaffold to the target ligand can be increased by the influence of the multi-valent interaction, so that the polyproline nanocage compound of the present disclosure can selectively recognize cyclodextrins, and it is favorable for being an artificial receptor through multivalent recognition.

In summary, the advantages of the polyproline nanocage compound of the present disclosure are as below. Firstly, the polyproline helix rods are used as the assembling units with the biocompatibility, low toxicity and stability so as to be applied to biomedical applications. Secondly, the polyproline nanocage compound is formed by connecting the cyclic peptide scaffolds with covalent bonds, and the polyproline nanocage compound is highly stable in a complicated environment. Thirdly, the polyproline nanocage compound is assembled via a stepwise assembly, and the connection order of each unit can be controlled arbitrarily, so that a polyproline nanocage compound with an asymmetric structure can be formed. Fourthly, the reacting molecules connected to the polyproline nanocage compound at specific positions can be controlled by using various protecting groups. Fifthly, due to the characteristics of the structure of the polyproline helix rods, the size of the polyproline nanocage compound can be controlled by controlling the scaffold size so as to host different guest molecules, and has a potential to be an artificial receptor.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A polyproline nanocage compound, comprising:
two cyclic peptide scaffolds, and each of the two cyclic peptide scaffolds comprising:
at least three polyproline helix rods, wherein each of the at least three polyproline helix rods is composed of a plurality of repeat units, and each of the plurality of repeat units is represented by Formula (i): 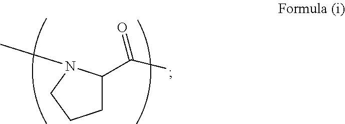
and
at least three turn-angle molecules, wherein two of the at least three polyproline helix rods are connected by one of the at least three turn-angle molecules so as to form a closed ring; and
at least three connecting molecules, wherein the at least three connecting molecules are respectively connected from the at least three polyproline helix rods of one of the two cyclic peptide scaffolds to the at least three polyproline helix rods of the other of the two cyclic peptide scaffolds.

2. The polyproline nanocage compound of claim 1, wherein a number of the plurality of repeat units of each of the at least three polyproline helix rods of each of the two cyclic peptide scaffolds is 4 to 12.

3. The polyproline nanocage compound of claim 1, wherein each of the at least three turn-angle molecules of each of the two cyclic peptide scaffolds has a structure represented by Formula (ii): 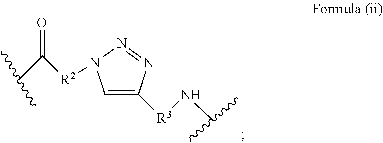
wherein, R² and R³ are independently a single bond or a divalent hydrocarbon group having 1 to 6 carbon atoms.

4. The polyproline nanocage compound of claim 2, wherein at least one of the plurality of repeat units of at least one of the at least three polyproline helix rods of each of the two cyclic peptide scaffolds is connected to a functional group.

5. The polyproline nanocage compound of claim 1, wherein the closed ring is a triangle, a quadrangle, a pentagon, a hexagon, a heptagon or an octagon.

6. A polyproline nanocage compound, comprising:
a cyclic peptide scaffold, comprising:
at least three polyproline helix rods, wherein each of the at least three polyproline helix rods is composed of a plurality of repeat units, and each of the plurality of repeat units is represented by Formula (i): 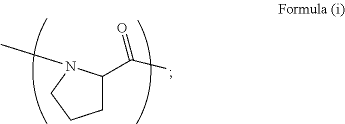
and
at least three turn-angle molecules, wherein two of the at least three polyproline helix rods are connected by one of the at least three turn-angle molecules so as to form a closed ring; and
at least three connecting molecules, wherein one terminus of each of the at least three connecting molecules is connected to one of the at least three polyproline helix rods of the cyclic peptide scaffold, and the other terminus of each of the at least three connecting molecules is connected to the other terminus of another of the at least three connecting molecules.

7. The polyproline nanocage compound of claim 6, wherein a number of the plurality of repeat units of each of the at least three polyproline helix rods is 4 to 12.

8. The polyproline nanocage compound of claim 6, wherein each of the at least three turn-angle molecules has a structure represented by Formula (ii): 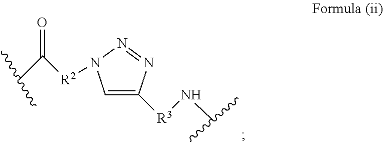
wherein, R² and R³ are independently a single bond or a divalent hydrocarbon group having 1 to 6 carbon atoms.

9. The polyproline nanocage compound of claim 7, wherein at least one of the plurality of repeat units of at least one of the at least three polyproline helix rods is connected to a functional group.

10. The polyproline nanocage compound of claim 6, wherein the closed ring is a triangle, a quadrangle, a pentagon, a hexagon, a heptagon or an octagon.

11. A manufacturing method of a polyproline nanocage compound, comprising:
providing two cyclic peptide scaffolds, wherein each of the two cyclic peptide scaffolds comprises:
at least three polyproline helix rods, wherein each of the at least three polyproline helix rods is composed of a plurality of repeat units, and each of the plurality of repeat units is represented by Formula (i): 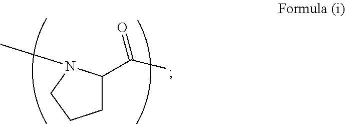
at least three turn-angle molecules, wherein two of the at least three polyproline helix rods are connected by one of the at least three turn-angle molecules so as to form a closed ring; and
at least three modifying functional groups, wherein the at least three modifying functional groups are respectively connected to the at least three polyproline helix rods, and each of the at least three modifying functional groups is formed by protecting an amino group with a protecting group;
performing a de-protecting step, wherein the protecting group of one of the at least three modifying functional groups of each of the two cyclic peptide scaffolds is removed to form a first primary amine, and an azide compound is added so that the first primary amine is converted into a first azido group;
performing a first cycloaddition, wherein a first connecting intermediate with an alkynyl group at one terminus and with a carboxyl group at the other terminus is catalyzed via a Cu ion, so that the alkynyl group of the first connecting intermediate is connected to the first azido group of one of the two cyclic peptide scaffolds;
performing a second cycloaddition, wherein the carboxyl group of the first connecting intermediate is modified to form an alkynyl terminus, so that the alkynyl terminus is connected to the first azido group of the other of the two cyclic peptide scaffolds; and
performing a connecting step, wherein the protecting groups of another at least two of the at least three modifying functional groups of each of the two cyclic peptide scaffolds are removed to respectively form at least two second primary amines, the at least two second primary amines are converted into at least two second azido groups, at least two second connecting intermediates with the alkynyl groups at two termini are catalyzed via the Cu ion, so that the at least two second azido groups of each of the two cyclic peptide scaffolds are connected to the alkynyl groups of the at least two second connecting intermediates so as to form a polyproline nanocage compound;
wherein at least three connecting molecules are respectively formed from the first connecting intermediate after performing the second cycloaddition and from the at least two second connecting intermediates after performing the connecting step.

12. The manufacturing method of the polyproline nanocage compound of claim 11, wherein a number of the plurality of repeat units of each of the at least three polyproline helix rods of each of the two cyclic peptide scaffolds is 4 to 12.

13. The manufacturing method of the polyproline nanocage compound of claim 12, wherein at least one of the plurality of repeat units of at least one of the at least three polyproline helix rods of each of the two cyclic peptide scaffolds is connected to a functional group.

14. The manufacturing method of the polyproline nanocage compound of claim 11, wherein the protecting group of each of the at least three modifying functional groups is independently an allyloxycarbonyl protecting group, a tert-butoxycarbonyl protecting group or a benzyloxycarbonyl protecting group.

15. A manufacturing method of a polyproline nanocage compound, comprising:
providing a cyclic peptide scaffold, wherein the cyclic peptide scaffold comprises:
at least three polyproline helix rods, wherein each of the at least three polyproline helix rods is composed of a plurality of repeat units, and each of the plurality of repeat units is represented by Formula (i): 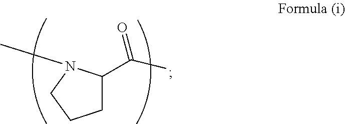
at least three turn-angle molecules, wherein two of the at least three polyproline helix rods are connected by one of the at least three turn-angle molecules so as to form a closed ring; and
at least three modifying functional groups, wherein the at least three modifying functional groups are respectively connected to the at least three polyproline helix rods, and each of the at least three modifying functional groups is formed by protecting an amino group with a protecting group;
providing at least three first connecting intermediates, wherein each of the at least three first connecting intermediates is with an alkynyl group at one terminus and with a carboxyl group at the other terminus;
performing a first de-protecting step, wherein the protecting group of one of the at least three modifying functional groups is removed to form a first primary amine, and an azide compound is added so that the first primary amine is converted into a first azido group;
performing a first cycloaddition, wherein one of the at least three first connecting intermediates is catalyzed via a Cu ion, so that the alkynyl group of the one of the at least three first connecting intermediates is connected to the first azido group of the cyclic peptide scaffold;
performing a second de-protecting step, wherein the protecting groups of another at least two of the at least three modifying functional groups of the cyclic peptide scaffold are removed to form at least two second primary amines, so that the at least two second primary amines are converted into at least two second azido groups;
performing a second cycloaddition, wherein another at least two of the at least three first connecting intermediates are catalyzed via the Cu ion, so that the alkynyl groups of the another at least two of the at least three first connecting intermediates are connected to the at least two second azido groups of the cyclic peptide scaffold; and
performing a connecting step, wherein the carboxyl groups of the at least three first connecting intermediates are modified to azido groups, and are connected to each other so as to form a polyproline nanocage compound;
wherein at least three first connecting molecules are formed from the at least three first connecting intermediates after performing the connecting step.

16. The manufacturing method of the polyproline nanocage compound of claim 15, wherein a number of the plurality of repeat units of each of the at least three polyproline helix rods is 4 to 12.

17. The manufacturing method of the polyproline nanocage compound of claim 16, wherein at least one of the plurality of repeat units of at least one of the at least three polyproline helix rods is connected to a functional group.

18. The manufacturing method of the polyproline nanocage compound of claim 15, wherein the protecting group of each of the at least three modifying functional groups is independently an allyloxycarbonyl protecting group, a tert-butoxycarbonyl protecting group or a benzyloxycarbonyl protecting group.
